(19) United States
(12) Patent Application Publication
(10) Pub. No.: US 2018/0093956 A1
(43) Pub. Date: Apr. 5, 2018

(54) SUBSTITUTED PHTHALAZINES

(71) Applicant: AbbVie Inc., North Chicago, IL (US)

(72) Inventors: Yujia Dai, Gurnee, IL (US); Michael Michaelides, Libertyville, IL (US)

(21) Appl. No.: 15/549,090

(22) PCT Filed: Feb. 6, 2015
(86) PCT No.: PCT/CN2015/072421

§ 371 Date: Aug. 4, 2017

(51) Int. Cl. C07D 237/34; C07D 401/04; C07D 403/04; C07D 409/04; C07D 401/14; C07D 413/14; C12Q 1/48

(52) U.S. Cl. CPC C07D 237/34; C07D 401/04; C07D 403/04; C12Q 1/48; C07D 401/14; C07D 413/14; C07D 409/04

(57) ABSTRACT

The invention provides for compounds of formula (I)

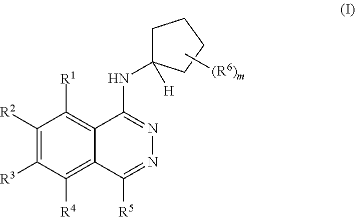

wherein R¹, R², R³, R⁴, R⁵, and R⁶ have any of the values defined in the specification, and pharmaceutically acceptable salts thereof, that are useful as agents in the treatment of diseases and conditions mediated and modulated by SUV420H1. Also provided are pharmaceutical compositions comprised of one or more compounds of formula (I).

### BACKGROUND

### Technical Field

Histone methyltransferases (HMTs), a class of enzymatic “writers” of epigenetic marks, have recently emerged as targets of potential therapeutic value. They catalyze the methylation of histone lysines and arginines utilizing S-adenosyl-methionine (SAM) as the cofactor/methyl-source. This process can result in either the activation or repression of transcription. Dysregulation of methylation at specific histone sites (alterations in the “histone code”) has been implicated in many cancers such as breast cancers, prostate cancers, renal cell carcinoma, and myeloid and lymphoblastic leukaemia (Chi P. et al. (2010) Nat. Rev. Cancer 10, 457-469). Hence, targeting HMT activity has been the subject of much investigation in the field of oncology.

Suppressor of variegation 4-20 homolog 1 (SUV420H1) is a SET domain-containing histone methyltransferase that localizes to heterochromatin (Schotta G. et al. (2004) Genes & development 18: 1251-1262). There are two isoforms of SUV420H1, both of which contain the conserved SET domain but differ at their C-termini, as well as a closely related homolog, SUV420H2 (Tsang L. W. et al. (2010) PloS one 5: e14447). SUV420H1 binds heterochromatin protein 1 (HP1) and this interaction functions to recruit SUV420H1 to heterochromatin. SUV420H1 catalyzes the di-methylation of histone H4 at lysine 20 (H4K20), which mediates a number of biological processes, including DNA replication, DNA damage-induced stress signaling, and the maintenance of pericentric and telomeric heterochromatin (Schotta G. et al. (2004) Genes & development 18: 1251-1262; Benetti R. et al. (2007) The Journal of cell biology 178: 925-936; Schotta G. et al. (2008) Genes & development 22: 2048-2061; Beck D. B. et al. (2012) Genes & development 26: 2580-2589; Kuo A. J. et al. (2012) Nature 484: 115-119; Tuzon C. T. et al. (2014) Cell reports 8: 430-438).

There is an increasing body of evidence indicating SUV420H1 plays a key role in cell growth and proliferation, and may be associated with proliferative diseases such as cancers. SUV420H1 knockout animals show embryonic and perinatal lethality (Schotta G. et al. (2008) Genes & development 22: 2048-2061). Furthermore, SUV420H1-deficient cells show reduced proliferation rates and growth arrest/senescence due to defects in DNA replication during S phase (Schotta G. et al. (2008) Genes & development 22: 2048-2061). Mechanistically, these defects in DNA replication arise from the lack of SUV420H1-dependent H4K20 dimethylation and subsequent inhibition of replication origin licensing (Beck D. B. et al. (2012) Genes & development 26: 2580-2589; Kuo A. J. et al. (2012) Nature 484: 115-119). Indeed, proteins involved in replication origin licensing have been pursued as targets for cancer therapy (Lei M. (2005) Current cancer drug targets 5: 365-380; Zimmerman K. M. et al. (2013) Molecular cancer research: MCR 11: 370-380). SUV420H1 is also important in regulating non-homologous end joining processes during the DNA damage response to double strand breaks (Schotta G. et al. (2008) Genes & development 22: 2048-2061; Tuzon C. T. et al. (2014) Cell reports 8: 430-438). In particular, inhibition of non-homologous end joining processes has been shown to sensitize pancreatic, breast, cervical and colon cancer cells to DNA damaging agents (Zhao Y. et al. (2006) Cancer research 66: 5354-5362; Chen X. et al. (2008) Cancer research 68: 3169-3177; Li Y. H. et al. (2012) PloS one 7: e39588).

Currently no small molecule inhibitors of SUV420H1 have been reported. Accordingly, there is a need for novel compounds able to inhibit SUV420H1.

### SUMMARY

In one aspect the invention provides for compounds of formula (I)

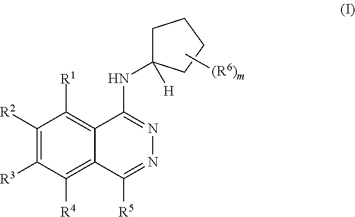

or a pharmaceutically acceptable salt thereof, wherein

R¹ and R⁴ are each independently H, halogen, CN, C₁-C₃ alkyl, C₁-C₃ haloalkyl, —O—(C₁-C₃ alkyl), or —O—(C₁-C₃ haloalkyl);

R² and R³ are each independently halogen, CN, C₁-C₃ alkyl, C₁-C₃ haloalkyl, —O—(C₁-C₃ alkyl), or —O—(C₁-C₃ haloalkyl);

R⁵ is halogen, -G¹, —C(O)G², —C(O)O(R^{A}), or —C(O)N(R^{A})(R^{B});

G¹ is aryl, heteroaryl, or heterocycle, each of which is optionally substituted with 1, 2, or 3 R^{u} groups;

R^{u}, at each occurrence, is independently C₁-C₆ alkyl, C₂-C₆ alkenyl, C₂-C₆ alkynyl, halogen, C₁-C₆ haloalkyl, —CN, oxo, NO₂, —ORS, —OC(O)R^{k}, —OC(O)N(R^{j})₂, —SR^{j}, —S(O)₂R^{j}, —S(O)₂N(R^{j})₂, —C(O)R^{j}, —C(O)OR^{j}, —C(O)N(R^{j})₂, —N(R^{j})₂, —N(R^{j})C(O)R^{k}, —N(R^{j})S(O)₂R^{k}, —N(R^{j})C(O)O(R^{k}), —N(R^{j})C(O)N(R^{j})₂, -G^{1A}, —(C₁-C₆ alkylenyl)-OR^{j}, —(C₁-C₆ alkylenyl)-OC(O)R^{k}, —(C₁-C₆ alkylenyl)-OC(O)N(R^{j})₂, —(C₁-C₆ alkylenyl)-SR^{j}, —(C₁-C₆ alkylenyl)-S(O)₂R^{j}, —(C₁-C₆ alkylenyl)-S(O)₂N(R^{j})₂, —(C₁-C₆ alkylenyl)-C(O)R^{j}, —(C₁-C₆ alkylenyl)-C(O)OR^{j}, —(C₁-C₆ alkylenyl)-C(O)N(R^{j})₂, —(C₁-C₆ alkylenyl)-N(R^{j})₂, —(C₁-C₆ alkylenyl)-N(R^{j})C(O)R^{k}, —(C₁-C₆ alkylenyl)-N(R^{j})S(O)2R^{k}, —(C₁-C₆ alkylenyl)-N(R^{j})C(O)O(R^{k}), —(C₁-C₆ alkylenyl)-N(R^{j})C(O)N(R^{j})₂, —(C₁-C₆ alkylenyl)-CN, or —(C₁-C₆ alkylenyl)-G^{1A};

R^{j}, at each occurrence, is independently hydrogen, C₁-C₆ alkyl, C₁-C₆ haloalkyl, -G^{1A}, —(C₁-C₆ alkylenyl)-OR^{m}, —(C₁-C₆ alkylenyl)-CN, —(C₁-C₆ alkylenyl)-S(O)₂R^{m}, or —(C₁-C₆ alkylenyl)-C(O)N(R^{m})₂;

R^{k}, at each occurrence, is independently C₁-C₆ alkyl, C₁-C₆ haloalkyl, -G^{1A}, —(C₁-C₆ alkylenyl)-OR^{m}, —(C₁-C₆ alkylenyl)-CN, —(C₁-C₆ alkylenyl)-S(O)₂R^{m}, or —(C₁-C₆ alkylenyl)-C(O)N(R^{m})₂;

G^{1A}, at each occurrence, is independently aryl, cycloalkyl, heteroaryl, or heterocycle, each of which is optionally substituted with 1, 2, or 3 R^{v} groups;

G² is a monocyclic heterocycle which is optionally substituted with 1, 2, or 3 R^{v} groups;

R^{A}, at each occurrence, is independently H, C₁-C₆ alkyl, or C₁-C₆ haloalkyl;

R^{B} is H, C₁-C₆ alkyl, C₁-C₆ haloalkyl, C₁-C₆ hydroxyalkyl, phenyl, or monocyclic heteroaryl; wherein the phenyl and the monocyclic heteroaryl are optionally substituted with 1, 2, or 3 R^{v} groups;

R^{v}, at each occurrence, is independently C₁-C₆ alkyl, C₂-C₆ alkenyl, C₂-C₆ alkynyl, halogen, C₁-C₆ haloalkyl, —CN, oxo, NO₂, —OR^{m}, —OC(O)Rⁿ, —OC(O)N(R^{m})₂, —S(O)₂R^{m}, —S(O)₂N(R^{m})₂, —C(O)R^{m}, —C(O)OR^{m}, —C(O)N(R^{m})₂, —N(R^{m})₂, —N(R^{m})C(O)Rⁿ, —N(R^{m})S(O)₂Rⁿ, —N(R^{m})C(O)O(Rⁿ), —N(R^{m})C(O)N(Rⁿ)₂, —(C₁-C₆ alkylenyl)-OR^{m}, —(C₁-C₆ alkylenyl)-OC(O)Rⁿ, —(C₁-C₆ alkylenyl)-OC(O)N(R^{m})₂, —(C₁-C₆ alkylenyl)-SRⁿ, —(C₁-C₆ alkylenyl)-S(O)₂R^{m}, —(C₁-C₆ alkylenyl)-S(O)₂N(R^{m})₂, —(C₁-C₆ alkylenyl)-C(O)R^{m}, —(C₁-C₆ alkylenyl)-C(O)OR^{m}, —(C₁-C₆ alkylenyl)-C(O)N(R^{m})₂, —(C₁-C₆ alkylenyl)-N(R^{m})₂, —(C₁-C₆ alkylenyl)-N(R^{j})C(O)Rⁿ, —(C₁-C₆ alkylenyl)-N(R^{j})S(O)₂Rⁿ, —(C₁-C₆ alkylenyl)-N(R^{m})C(O)O(Rⁿ), (C₁-C₆ alkylenyl)-N(R^{m})C(O)N(Rⁿ)₂, or —(C₁-C₆ alkylenyl)-CN;

R^{m}, at each occurrence, is independently hydrogen, C₁-C₆ alkyl, or C₁-C₆ haloalkyl;

Rⁿ, at each occurrence, is independently C₁-C₆ alkyl or C₁-C₆ haloalkyl;

R⁶ are optional substituents and at each occurrence, are each independently halogen, CN, C₁-C₃ alkyl, C₁-C₃ haloalkyl, —OH, —O—(C₁-C₃ alkyl), —O—(C₁-C₃ haloalkyl), or —S(O)₂—(C₁-C₃ alkyl); and

m is 0, 1, 2, or 3.

Another aspect of the invention relates to pharmaceutical compositions comprising compounds of the invention.

The compounds, compositions comprising the compounds, and methods for making the compounds are further described herein.

These and other objects of the invention are described in the following paragraphs. These objects should not be deemed to narrow the scope of the invention.

### DETAILED DESCRIPTION OF THE INVENTION

Described herein are compounds of formula (I)

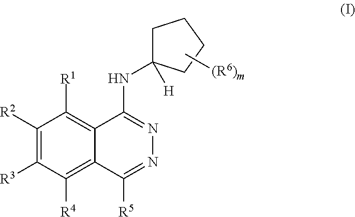

wherein R¹, R², R³, R⁴, R⁵, and R⁶ are defined above in the Summary of the Invention and below in the Detailed Description. Further, compositions comprising such compounds and methods for making such compounds are also included.

Compounds included herein may contain one or more variable(s) that occur more than one time in any substituent or in the formulae herein. Definition of a variable on each occurrence is independent of its definition at another occurrence. Further, combinations of substituents are permissible only if such combinations result in stable compounds. Stable compounds are compounds, which can be isolated from a reaction mixture.

### Definitions

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds, reference to “optionally a pharmaceutically acceptable carrier” refers to a single optional pharmaceutically acceptable carrier as well as one or more pharmaceutically acceptable carriers, and the like.

As used in the specification and the appended claims, unless specified to the contrary, the following terms have the meaning indicated:

The term “alkenyl” as used herein, means a straight or branched hydrocarbon chain containing from 2 to 10 carbons and containing at least one carbon-carbon double bond. The term “C₂-C₆ alkenyl” or “C₂-C₄ alkenyl” means an alkenyl group containing 2-6 carbon atoms or 2-4 carbon atoms respectively. Non-limiting examples of alkenyl include buta-1,3-dienyl, ethenyl, 2-propenyl, 2-methyl-2-propenyl, 3-butenyl, 4-pentenyl, and 5-hexenyl.

The term “alkyl” as used herein, means a saturated, straight or branched hydrocarbon chain radical. In some instances, the number of carbon atoms in an alkyl moiety is indicated by the prefix “Cₓ-C_{y}”, wherein x is the minimum and y is the maximum number of carbon atoms in the substituent. Thus, for example, “C₁-C₆ alkyl” means an alkyl substituent containing from 1 to 6 carbon atoms and “C₁-C₃ alkyl” means an alkyl substituent containing from 1 to 3 carbon atoms. Representative examples of C₁-C₆ alkyl include, but are not limited to, methyl, ethyl, n-propyl, iso-propyl, n-butyl, sec-butyl, iso-butyl, tert-butyl, n-pentyl, isopentyl, neopentyl, n-hexyl, 1-methylbutyl, 2-methylbutyl, 3-methylbutyl, 3,3-dimethylbutyl, 1,1-dimethylpropyl, 1,2-dimethylpropyl, 2,2-dimethylpropyl, 1-methylpropyl, 2-methylpropyl, 1-ethylpropyl, and 1,2,2-trimethylpropyl.

The term “alkylene” or “alkylenyl” means a divalent radical derived from a straight or branched, saturated hydrocarbon chain, for example, of 1 to 10 carbon atoms or of 1 to 6 carbon atoms (C₁-C₆ alkylenyl) or of 1 to 4 carbon atoms or of 1 to 3 carbon atoms (C₁-C₃ alkylenyl) or of 2 to 6 carbon atoms (C₂-C₆ alkylenyl). Examples of C₁-C₆ alkylenyl include, but are not limited to, —CH₂—, —CH₂CH₂—, —C((CH₃)₂)—CH₂CH₂CH₂—, —C((CH₃)₂)—CH₂CH₂, —CH₂CH₂CH₂CH₂—, and —CH₂CH(CH₃)CH₂—.

The term “C₂-C₆ alkynyl” as used herein, means a straight or branched chain hydrocarbon radical containing from 2 to 6 carbon atoms and containing at least one carbon-carbon triple bond. Representative examples of C₂-C₆ alkynyl include, but are not limited, to acetylenyl, 1-propynyl, 2-propynyl, 3-butynyl, 2-pentynyl, and 1-butynyl.

The term “aryl” as used herein, means phenyl or a bicyclic aryl. The bicyclic aryl is naphthyl, or a phenyl fused to a monocyclic cycloalkyl, or a phenyl fused to a monocyclic cycloalkenyl. Non-limiting examples of the aryl groups include dihydroindenyl, indenyl, naphthyl, dihydronaphthalenyl, and tetrahydronaphthalenyl. The monocyclic and the bicyclic aryls, including the exemplary rings, are optionally substituted unless otherwise indicated. The bicyclic aryls are attached to the parent molecular moiety through any carbon atom contained within the bicyclic ring systems.

The term “cycloalkyl” as used herein, means a radical that is a monocyclic cycloalkyl or a bicyclic cycloalkyl. The monocyclic cycloalkyl is a carbocyclic ring system containing three to eight carbon atoms, zero heteroatoms and zero double bonds. Examples of monocyclic cycloalkyl include cyclopropyl, cyclobutyl, cyclopentyl, cyclohexyl, cycloheptyl, and cyclooctyl. The bicyclic cycloalkyl is a monocyclic cycloalkyl fused to a monocyclic cycloalkyl ring. The monocyclic and the bicyclic cycloalkyl groups may contain one or two alkylene bridges, each consisting of one, two, three, or four carbon atoms in length, and each bridge links two non-adjacent carbon atoms of the ring system. Non-limiting examples of bicyclic ring systems include bicyclo[3.1.1]heptyl, bicyclo[2.2.1]heptyl, bicyclo[2.2.2]octyl, bicyclo[1.1.1]pentyl, bicyclo[3.2.2]nonyl, bicyclo[3.3.1]nonyl, bicyclo[4.2.1]nonyl, tricyclo[3.3.1.0^{3,7}]nonyl (octahydro-2,5-methanopentalene or noradamantyl), and tricyclo[3.3.1.1^{3,7}]decane (adamantyl). The monocyclic and the bicyclic cycloalkyls, including exemplary rings, are optionally substituted unless otherwise indicated. The monocyclic cycloalkyl and the bicyclic cycloalkyl are attached to the parent molecular moiety through any substitutable carbon atom contained within the ring systems.

The term “C₃-C₆ cycloalkyl” as used herein, means cyclopropyl, cyclobutyl, cyclopentyl, and cyclohexyl, each of which is optionally substituted unless otherwise indicated.

The term “cycloalkenyl” as used herein, refers to a monocyclic or a bicyclic hydrocarbon ring radical. The monocyclic cycloalkenyl has four-, five-, six-, seven- or eight carbon atoms and zero heteroatoms. The four-membered ring systems have one double bond, the five-or six-membered ring systems have one or two double bonds, and the seven- or eight-membered ring systems have one, two, or three double bonds. Representative examples of monocyclic cycloalkenyl groups include, but are not limited to, cyclobutenyl, cyclopentenyl, cyclohexenyl, cycloheptenyl, and cyclooctenyl. The bicyclic cycloalkenyl is a monocyclic cycloalkenyl fused to a monocyclic cycloalkyl group, or a monocyclic cycloalkenyl fused to a monocyclic cycloalkenyl group. The monocyclic and bicyclic cycloalkenyl ring may contain one or two alkylene bridges, each consisting of one, two, or three carbon atoms, and each linking two non-adjacent carbon atoms of the ring system. Representative examples of the bicyclic cycloalkenyl groups include, but are not limited to, 4,5,6,7-tetrahydro-3aH-indene, octahydronaphthalenyl, and 1,6-dihydro-pentalene. The monocyclic and the bicyclic cycloalkenyls, including exemplary rings, are optionally substituted unless otherwise indicated. The monocyclic cycloalkenyl and bicyclic cycloalkenyl are attached to the parent molecular moiety through any substitutable atom contained within the ring systems.

The term “halo” or “halogen” as used herein, means Cl, Br, I, and F.

The term “haloalkyl” as used herein, means an alkyl group, as defined herein, in which one, two, three, four, five or six hydrogen atoms are replaced by halogen. The term “C₁-C₆ haloalkyl” means a C₁-C₆ alkyl group, as defined herein, in which one, two, three, four, five, or six hydrogen atoms are replaced by halogen. The term “C₁-C₃ haloalkyl” means a C₁-C₃ alkyl group, as defined herein, in which one, two, three, four, or five hydrogen atoms are replaced by halogen. Representative examples of C₁-C₆ haloalkyl include, but are not limited to, chloromethyl, 2-fluoroethyl, 2,2-difluoroethyl, fluoromethyl, 2,2,2-trifluoroethyl, trifluoromethyl, difluoromethyl, pentafluoroethyl, 2-chloro-3-fluoropentyl, trifluorobutyl, and trifluoropropyl.

The term “heterocycle” or “heterocyclic” as used herein, means a radical of a monocyclic heterocycle and a bicyclic heterocycle ring structure that may be saturated (i.e. heterocycloalkyl) or partially saturated (i.e. heterocycloalkenyl). A monocyclic heterocycle is a three-, four-, five-, six-, seven-, or eight-membered carbocyclic ring wherein at least one carbon atom is replaced by heteroatom independently selected from the group consisting of O, N, and S. A three- or four-membered ring contains zero or one double bond, and one heteroatom selected from the group consisting of O, N, and S. A five-membered ring contains zero or one double bond and one, two, or three heteroatoms selected from the group consisting of O, N, and S. Examples of five-membered heterocyclic rings include those containing in the ring: 1 O; 1 S; 1 N; 2 N; 3 N; 1 S and 1 N; 1 S, and 2 N; 1 O and 1 N; or 1 O and 2 N. Non limiting examples of 5-membered heterocyclic groups include 1,3-dioxolanyl, tetrahydrofuranyl, dihydrofuranyl, tetrahydrothienyl, dihydrothienyl, imidazolidinyl, oxazolidinyl, imidazolinyl, isoxazolidinyl, pyrazolidinyl, pyrazolinyl, pyrrolidinyl, 2-pyrrolinyl, 3-pyrrolinyl, thiazolinyl, and thiazolidinyl. A six-membered ring contains zero, one, or two double bonds and one, two, or three heteroatoms selected from the group consisting of O, N, and S. Examples of six-membered heterocyclic rings include those containing in the ring: 1 O; 2 O; 1 S; 2 S; 1 N; 2 N; 3 N; 1 S, 1 O, and 1 N; 1 S and 1 N; 1 S and 2 N; 1 S and 1 O; 1 S and 2 O; 1 Q and 1 N; and 1 O and 2 N. Examples of 6-membered heterocyclic groups include tetrahydropyranyl, dihydropyranyl, dioxanyl, 1,4-dithianyl, hexahydropyrimidine, morpholinyl, piperazinyl, piperidinyl, 1,2,3,4-tetrahydropyridinyl, 1,2,3,6-tetrahydropyridinyl, tetrahydrothiopyranyl, thiomorpholinyl, thioxanyl, and trithianyl. Seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyridinyl, tetrahydropyranyl, tetrahydrothienyl, thiadiazolinyl, thiadiazolidinyl, thiazolinyl, thiazolidinyl, thiomorpholinyl, thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle. Representative examples of bicyclic heterocycles include, but are not limited to, benzopyranyl, benzothiopyranyl, 2,3-dihydrobenzofuranyl, 2,3-dihydrobenzothienyl, 2,3-dihydro-1H-indolyl, 3,4-dihydroisoquinolin-2(1H)-yl, 2,3,4,6-tetrahydro-1H-pyrido[1,2-a]pyrazin-2-yl, hexahydropyrano[3,4-b][1,4]oxazin-1(5H)-yl. The monocyclic heterocycle and the bicyclic heterocycle may contain one or two alkylene bridges, each consisting of no more than four carbon atoms and each linking two non-adjacent atoms of the ring system. Examples of such bridged heterocycle include, but are not limited to, azabicyclo[2.2.1]heptyl (including 2-azabicyclo[2.2.1]hept-2-yl), 8-azabicyclo[3.2.1]oct-8-yl, octahydro-2,5-epoxypentalene, hexahydro-2H-2,5-methanocyclopenta[b]furan, hexahydro-1H-1,4-methanocyclopenta[c]furan, aza-admantane (1-azatricyclo[3.3.1.1^{3,7}]decane), and oxa-adamantane (2-oxatricyclo[3.3.1.1^{3,7}]decane). The monocyclic and the bicyclic heterocycles, including exemplary rings, are optionally substituted unless otherwise indicated. The monocyclic and the bicyclic heterocycles are connected to the parent molecular moiety through any carbon atom or any nitrogen atom contained within the ring systems. The nitrogen and sulfur heteroatoms in the heterocycle rings may optionally be oxidized (e.g. 1,1-dioxidotetrahydrothienyl, 1,1-dioxido-1,2-thiazolidinyl, 1,1-dioxidothiomorpholinyl)) and the nitrogen atoms may optionally be quarternized.

The term “C₄-C₆ heterocycle” as used herein, means a 4, 5, or 6 membered monocyclic heterocycle as defined herein above. Examples of C₄-C₆ heterocycle include, but are not limited to, azetidinyl, pyrrolidinyl, tetrahydrofuranyl, tetrahydropyranyl, tetrahydropyridinyl, piperazinyl, piperidinyl, thiomorpholinyl, and morpholinyl. The C₄-C₆ heterocycle, including exemplary rings, are optionally substituted unless otherwise indicated.

The term “heteroaryl” as used herein, means a monocyclic heteroaryl and a bicyclic heteroaryl. The monocyclic heteroaryl is a five- or six-membered ring wherein at least one carbon atom is replaced by heteroatom independently selected from the group consisting of O, N, and S. The five-membered ring contains two double bonds. The five membered ring may contain one heteroatom selected from O or S; or one, two, three, or four nitrogen atoms and optionally one oxygen or one sulfur atom. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl consists of a monocyclic heteroaryl fused to a phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryls include, but are not limited to, benzofuranyl, benzothienyl, benzoxazolyl, benzimidazolyl, benzoxadiazolyl, benzothiazolyl, 1H-benzo[d][1,2,3]triazolyl, furo[3,2-b]pyridinyl, phthalazinyl, benzo[d][1,2,3]thiadiazole, 3H-imidazo[4,5-b]pyridinyl, imidazo[1,2-c]pyridinyl, 1H-pyrrolo[3,2-b]pyridinyl, 1H-pyrrolo[2,3-b]pyridinyl, 1H-pyrazolo[3,4-b]pyridinyl, indazolyl, indolyl, isoindolyl, isoxazolo[5,4-b]pyridinyl, isoquinolinyl, naphthyridinyl, pyridoimidazolyl, quinolinyl, thieno[3,2-b]pyridinyl, thiazolo[5,4-b]pyridin-2-yl, and thiazolo[5,4-d]pyrimidin-2-yl. The monocyclic and bicyclic heteroaryls, including exemplary rings, are optionally substituted unless otherwise indicated. The monocyclic and bicyclic heteroaryls are connected to the parent molecular moiety through any substitutable carbon atom or any substitutable nitrogen atom contained within the ring systems. The nitrogen atom in the heteroaryl rings may optionally be oxidized and may optionally be quarternized.

The term “C₅-C₆ heteroaryl” as used herein, means a 5- or 6-membered monocyclic heteroaryl as described above. Examples of C₅-C₆ heteroaryl include furanyl, thienyl, pyrazolyl, imidazolyl, 1,2,4-oxadiazolyl, 1,2,4-triazolyl, 1,3-thiazolyl, pyridinyl, pyrimidinyl, pyridazinyl, and pyrazinyl. The C₅-C₆ heteroaryls, including exemplary rings, are optionally substituted unless otherwise indicated.

The term “heteroatom” as used herein, means a nitrogen, oxygen, and sulfur.

The term “C₁-C₆ hydroxyalkyl” as used herein, means a C₁-C₆ alkyl group, as defined herein, in which one, two, or three hydrogen atoms are replaced by —OH. Representative examples of C₁-C₆ hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “oxo” as used herein, means a ═O group.

The term “radiolabel” refers to a compound of the invention in which at least one of the atoms is a radioactive atom or radioactive isotope, wherein the radioactive atom or isotope spontaneously emits gamma rays or energetic particles, for example alpha particles or beta particles, or positrons. Examples of such radioactive atoms include, but are not limited to, ³H (tritium), ¹⁴C, ¹¹C, ¹⁵O, ¹⁸F, ³⁵S, ¹²³I, and ¹²⁵I.

If a moiety is described as “substituted”, a non-hydrogen radical is in the place of hydrogen radical of any substitutable atom of the moiety. Thus, for example, a substituted heterocycle moiety is a heterocycle moiety in which at least one non-hydrogen radical is in the place of a hydrogen radical on the heterocycle. It should be recognized that if there are more than one substitution on a moiety, each non-hydrogen radical may be identical or different (unless otherwise stated).

If a moiety is described as being “optionally substituted,” the moiety may be either (1) not substituted or (2) substituted. If a moiety is described as being optionally substituted with up to a particular number of non-hydrogen radicals, that moiety may be either (1) not substituted; or (2) substituted by up to that particular number of non-hydrogen radicals or by up to the maximum number of substitutable positions on the moiety, whichever is less. Thus, for example, if a moiety is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

Unless otherwise indicated, the terms C₁-C₆ alkyl, C₁-C₃ alkyl, C₁-C₆ haloalkyl, and C₁-C₃ haloalkyl are not further substituted.

The terms “treat”, “treating”, and “treatment” refer to a method of alleviating or abrogating a disease and/or its attendant symptoms.

The terms “prevent”, “preventing”, and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

The phrase “therapeutically effective amount” means an amount of a compound, or a pharmaceutically acceptable salt thereof, sufficient to prevent the development of or to alleviate to some extent one or more of the symptoms of the condition or disorder being treated when administered alone or in conjunction with another therapeutic agent or treatment in a particular subject or subject population. For example in a human or other mammal, a therapeutically effective amount can be determined experimentally in a laboratory or clinical setting, or may be the amount required by the guidelines of the United States Food and Drug Administration, or equivalent foreign agency, for the particular disease and subject being treated.

The term “subject” is defined herein to refer to animals such as mammals, including, but not limited to, primates (e.g., humans), cows, sheep, goats, pigs, horses, dogs, cats, rabbits, rats, mice and the like. In preferred embodiments, the subject is a human.

### Compounds

Compounds of the invention have the general formula (I) as described above.

Particular values of variable groups are as follows. Such values may be used where appropriate with any of the other values, definitions, claims or embodiments defined hereinbefore or hereinafter.

In certain embodiments, R¹ and R⁴ are H.

In certain embodiments, one of R² and R³ is halogen. In some such embodiments, the halogens are independently Cl, Br, or F. In some such embodiments, one of R² and R³ is Cl.

In certain embodiments, R² and R³ are halogen. In some such embodiments, the halogens are independently Cl, Br, or F. In some such embodiments, R² and R³ are Cl.

In certain embodiments, R⁵ is -G¹, —C(O)G², —C(O)O(R^{A}), or —C(O)N(R^{A})(R^{B}).

In certain embodiments, R⁵ is -G¹.

In certain embodiments, R⁵ is -G¹ wherein G¹ is phenyl, C₅-C₆ heteroaryl, or C₄-C₆ heterocycle, each of which is optionally substituted with 1, 2, or 3 R^{u} groups.

In certain embodiments, R⁵ is -G¹ wherein G¹ is phenyl which is optionally substituted with 1, 2, or 3 R^{u} groups. In some such embodiments, G¹ is unsubstituted phenyl.

In certain embodiments, R⁵ is -G¹ wherein -G¹ is C₅-C₆ heteroaryl which is optionally substituted with 1, 2, or 3 R^{u} groups.

In certain embodiments, R⁵ is -G¹ wherein G¹ is thienyl, pyridinyl, or pyrazolyl, each of which is optionally substituted with 1, 2, or 3 R^{u} groups.

In certain embodiments, R⁵ is -G¹ wherein -G¹ is pyridinyl which is optionally substituted with 1, 2, or 3 R^{u} groups.

In certain embodiments, R⁵ is -G¹ wherein -G¹ is pyrazolyl which is optionally substituted with 1, 2, or 3 R^{u} groups.

In certain embodiments, R⁵ is -G¹ wherein -G¹ is C₄-C₆ heterocycle which is optionally substituted with 1, 2, or 3 R^{u} groups.

In certain embodiments, R⁵ is -G¹ wherein -G¹ is pyrrolidinyl, piperidinyl, piperazinyl, morpholinyl, or tetrahydropyridinyl, each of which is optionally substituted with 1, 2, or 3 R^{u} groups.

In certain embodiments, each R^{u} is independently

C₁-C₆ alkyl,

halogen,

C₁-C₆ haloalkyl,

—S(O)₂R^{j} wherein is C₁-C₃ alkyl, optionally substituted phenyl, or optionally substituted cyclopropyl,

—C(O)R^{j} wherein R^{j} is G^{1A} wherein G^{1A} is phenyl, C₄-C₆ heterocycle, C₅-C₆ heteroaryl, or C₃-C₆ cycloalkyl, each of which is optionally substituted; or R^{j} is —(C₁-C₆ alkylenyl)-OR^{m}, —(C₁-C₆ alkylenyl)-CN, —(C₁-C₆ alkylenyl)-S(O)₂R^{m}, or —(C₁-C₆ alkylenyl)-C(O)N(R^{m})₂;

—N(R^{j})₂,

-G^{1A} wherein G^{1A} is optionally substituted C₄-C₆ heterocycle,

—(C₁-C₆ alkylenyl)-C(O)R^{j} wherein Rj is -G^{1A} and G^{1A} is optionally substituted C₄-C₆ heterocycle,

—(C₁-C₆ alkylenyl)-C(O)OR^{j} wherein R^{j} is hydrogen or C₁-C₆ alkyl,

—(C₁-C₆ alkylenyl)-C(O)N(R^{j})₂, or

—(C₁-C₆ alkylenyl)-G^{1A} wherein G^{1A} is optionally substituted C₄-C₆ heterocycle.

In certain embodiments, R⁵ is —C(O)G².

In certain embodiments, R⁵ is —C(O)G² wherein G² is pyrrolidinyl, piperazinyl, piperidinyl, morpholinyl, or thiomorpholinyl, each of which is optionally substituted with 1, 2, or 3 R^{v} groups. In some such embodiments, each R^{v} is independently C₁-C₆ alkyl, halogen, C₁-C₆ haloalkyl, —CN, oxo, or —OR^{m}.

In certain embodiments, R⁵ is —C(O)O(R^{A}). In some such embodiments, R^{A} is H or C₁-C₆ alkyl. In some such embodiments, R^{A} is H or methyl.

In certain embodiments, R⁵ is —C(O)N(R^{A})(R^{B}).

In certain embodiments, R⁵ is —C(O)N(R^{A})(R^{B}) wherein R^{A} is H or C₁-C₃ alkyl and R^{B} is H, C₁-C₆ alkyl, C₁-C₆ haloalkyl, C₁-C₆ hydroxyalkyl, pheny or pyrazolyl; wherein the phenyl and the pyrazolyl are optionally substituted with 1, 2, or 3 R^{v} groups. In some such embodiments, R^{v} is C₁-C₃ alkyl.

In certain embodiments, R⁶ is halogen, CH₃, CH₂F, or —OH.

In certain embodiments, m is 0 or 1.

In certain embodiments, m is 0 or 1, and R⁶ is —OH.

In certain embodiments, m is 0.

In certain embodiments, m is 1 and R⁶ is —OH.

Various embodiments of substituents R¹, R², R³, R⁴, R⁵, and R⁶ have been discussed above. These substituents embodiments can be combined to form various embodiments of the invention. All embodiments of present compounds, formed by combining the substituent embodiments discussed above are within the scope of Applicant's invention, and some illustrative embodiments of present compounds are provided below.

In one embodiment, the invention is directed to compounds of formula (I) wherein R¹ and R⁴ are H, and R² and R³ are halogen.

In one embodiment, the invention is directed to compounds of formula (I) wherein R¹ and R⁴ are H, and R² and R³ are Cl.

In one embodiment, the invention is directed to compounds of formula (I) wherein R² and R³ are halogen, m is 0 or 1, and R⁶ is halogen, CH₃, CH₂F, or OH. In some such embodiments, R² and R³ are Cl.

In one embodiment, the invention is directed to compounds of formula (I) wherein R² and R³ are halogen, and m is 0. In some such embodiments, R² and R³ are Cl.

In one embodiment, the invention is directed to compounds of formula (I) wherein R² and R³ are halogen, and m is 1, and R⁶ is —OH. In some such embodiments, R² and R³ are Cl.

In one embodiment, the invention is directed to compounds of formula (I) wherein R² and R³ are halogen, and R⁵ is -G¹, —C(O)G², —C(O)O(R^{A}), or —C(O)N(R^{A})(R^{B}). In some such embodiments, R² and R³ are Cl.

In one embodiment, the invention is directed to compounds of formula (I) wherein R² and R³ are halogen, R⁵ is -G¹, m is 0 or 1, and R⁶ is halogen, CH₃, CH₂F, or —OH.

In one embodiment, the invention is directed to compounds of formula (I) wherein R² and R³ are halogen, R⁵ is -G¹, and G¹ is phenyl, C₅-C₆ heteroaryl, or C₄-C₆ heterocycle, each of which is optionally substituted with 1, 2, or 3 R^{u} groups. In some such embodiments, R² and R³ are Cl.

In one embodiment, the invention is directed to compounds of formula (I) wherein R² and R³ are halogen, m is 0 or 1, R⁶ is halogen, CH₃, CH₂F, or —OH, R⁵ is -G¹, and G¹ is phenyl which is optionally substituted with 1, 2, or 3 R^{u} groups. In some such embodiments, R² and R³ are Cl. In some such embodiments, G¹ is unsubstituted phenyl.

In one embodiment, the invention is directed to compounds of formula (I) wherein R² and R³ are Cl, m is 0, R⁵ is -G¹, and G¹ is phenyl which is optionally substituted with 1, 2, or 3 R^{u} groups. In some such embodiments, G¹ is unsubstituted phenyl.

In one embodiment, the invention is directed to compounds of formula (I) wherein R¹ and R⁴ are H, R² and R³ are Cl, m is 0, R⁵ is -G¹, and G¹ is unsubstituted phenyl.

In one embodiment, the invention is directed to compounds of formula (I) wherein R² and R³ are halogen, m is 0 or 1, R⁶ is halogen, CH₃, CH₂F, or —OH, R⁵ is -G¹, and G¹ is C₅-C₆ heteroaryl which is optionally substituted with 1, 2, or 3 R^{u} groups. In some such embodiments, R² and R³ are Cl.

In one embodiment, the invention is directed to compounds of formula (I) wherein R² and R³ are Cl, m is 0, R⁵ is -G¹, and G¹ is thienyl, pyridinyl or pyrazolyl, each of which is optionally substituted with 1, 2, or 3 R^{u} groups.

In one embodiment, the invention is directed to compounds of formula (I) wherein R¹ and R⁴ are H, R² and R³ are Cl, m is 0, R⁵ is -G¹, and G¹ is pyridinyl or pyrazolyl, each of which is optionally substituted with 1, 2, or 3 R^{u} groups.

In one embodiment, the invention is directed to compounds of formula (I) wherein R² and R³ are halogen, m is 0 or 1, R⁶ is halogen, CH₃, CH₂F, or —OH, R⁵ is -G¹, and G¹ is C₄-C₆ heterocycle which is optionally substituted with 1, 2, or 3 R^{u} groups. In some such embodiments, R² and R³ are Cl.

In one embodiment, the invention is directed to compounds of formula (I) wherein R² and R³ are Cl, m is 0, R⁵ is -G¹, and G¹ is pyrrolidinyl, piperidinyl, piperazinyl, morpholinyl, or tetrahydropyridinyl, each of which is optionally substituted with 1, 2, or 3 R^{u} groups.

In one embodiment, the invention is directed to compounds of formula (I) wherein R¹ and R⁴ are H, R² and R³ are Cl, m is 0, R⁵ is -G¹, and G¹ is tetrahydropyridinyl which is optionally substituted with 1, 2, or 3 R^{u} groups.

In one embodiment, the invention is directed to compounds of formula (I) wherein R² and R³ are halogen, m is 0 or 1, R⁶ is halogen, CH₃, CH₂F, or —OH, and R⁵ is —C(O)G². In some such embodiments, R² and R³ are Cl.

In one embodiment, the invention is directed to compounds of formula (I) wherein R² and R³ are Cl, m is 0, R⁵ is —C(O)G² wherein G² is pyrrolidinyl, piperazinyl, piperidinyl, morpholinyl, or thiomorpholinyl, each of which is optionally substituted with 1, 2, or 3 R^{v} groups. In some such embodiments, each R^{v} is independently C₁-C₆ alkyl, halogen, C₁-C₆ haloalkyl, —CN, oxo, or —OR^{m}.

In one embodiment, the invention is directed to compounds of formula (I) wherein R¹ and R⁴ are H, R² and R³ are Cl, m is 0, R⁵ is —C(O)G² wherein G² is pyrrolidinyl, piperazinyl, piperidinyl, morpholinyl, or thiomorpholinyl, each of which is optionally substituted with 1, 2, or 3 R^{v} groups. In some such embodiments, each R^{v} is independently C₁-C₆ alkyl, halogen, C₁-C₆ haloalkyl, —CN, oxo, or —OR^{m}.

In one embodiment, the invention is directed to compounds of formula (I) wherein R² and R³ are halogen, m is 0 or 1, R⁶ is halogen, CH₃, CH₂F, or OH, and R⁵ is —C(O)OR^{A}. In some such embodiments, R² and R³ are Cl.

In one embodiment, the invention is directed to compounds of formula (I) wherein R¹ and R⁴ are H, R² and R³ are Cl, m is 0, R⁵ is —C(O)OR^{A}. In some such embodiments, R^{A} is H or C₁-C₆ alkyl. In some such embodiments, R^{A} is H or methyl.

In one embodiment, the invention is directed to compounds of formula (I) wherein R² and R³ are halogen, m is 0 or 1, R⁶ is halogen, CH₃, CH₂F, or —OH, and R⁵ is —C(O)N(R^{A})(R^{B}). In some such embodiments, R² and R³ are Cl.

In one embodiment, the invention is directed to compounds of formula (I) wherein R¹ and R⁴ are H, R² and R³ are Cl, m is 0, R⁵ is —C(O)N(R^{A})(R^{B}) wherein R^{A} is H or C₁-C₃ alkyl and R^{B} is H, C₁-C₆ alkyl, C₁-C₆ haloalkyl, C₁-C₆ hydroxyalkyl, pheny or pyrazolyl; wherein the phenyl and the pyrazolyl are optionally substituted with 1, 2, or 3 R^{v} groups. In some such embodiments, R^{v} is C₁-C₃ alkyl.

Exemplary compounds of formula (I) include, but are not limited to:

4,6,7-trichloro-N-cyclopentylphthalazin-1-amine;

6,7-dichloro-N-cyclopentyl-4-phenylphthalazin-1-amine;

6,7-dichloro-N-cyclopentyl-4-(pyridin-3-yl)phthalazin-1-amine;

6,7-dichloro-N-cyclopentyl-4-(1-methyl-1H-pyrazol-3-yl)phthalazin-1-amine;

6,7-dichloro-N-cyclopentyl-4-(1-methyl-1H-pyrazol-4-yl)phthalazin-1-amine;

6,7-dichloro-N-cyclopentyl-4-(pyridin-4-yl)phthalazin-1-amine;

6,7-dichloro-N-cyclopentyl-4-(thiophen-3-yl)phthalazin-1-amine;

6,7-dichloro-N-cyclopentyl-4-(piperidin-1-yl)phthalazin-1-amine;

6,7-dichloro-N-cyclopentyl-4-(4-methylpiperazin-1-yl)phthalazin-1-amine;

6,7-dichloro-N-cyclopentyl-4-(morpholin-4-yl)phthalazin-1-amine;

6,7-dichloro-N-cyclopentyl-4-(pyrrolidin-1-yl)phthalazin-1-amine;

6,7-dichloro-N-cyclopentyl-4-(2-fluoropyridin-4-yl)phthalazin-1-amine;

rac-(1R,3S)-3-[(4,6,7-trichlorophthalazin-1-yl)amino]cyclopentanol;

6,7-dichloro-N-cyclopentyl-4-[4-(morpholin-4-ylmethyl)phenyl]phthalazin-1-amine;

6,7-dichloro-N-cyclopentyl-4-[2-(pyrrolidin-1-yl)pyridin-4-yl]phthalazin-1-amine;

6,7-dichloro-N-cyclopentyl-4-[2-(4-methylpiperazin-1-yl)pyridin-4-yl]phthalazin-1-amine;

6,7-dichloro-N-cyclopentyl-4-[2-(morpholin-4-yl)pyridin-4-yl]phthalazin-1-amine;

6,7-dichloro-N-cyclopentyl-4-[2-(piperazin-1-yl)pyridin-4-yl]phthalazin-1-amine;

6,7-dichloro-N-cyclopentyl-4-(6-fluoropyridin-3-yl)phthalazin-1-amine;

6,7-dichloro-N-cyclopentyl-4-[6-(dimethylamino)pyridin-3-yl]phthalazin-1-amine;

6,7-dichloro-N-cyclopentyl-4-[6-(4-methylpiperazin-1-yl)pyridin-3-yl]phthalazin-1-amine;

6,7-dichloro-N-cyclopentyl-4-[6-(morpholin-4-yl)pyridin-3-yl]phthalazin-1-amine;

tert-butyl 4-[6,7-dichloro-4-(cyclopentylamino)phthalazin-1-yl]-3,6-dihydropyridine-1(2H)-carboxylate;

6,7-dichloro-N-cyclopentyl-4-(1,2,3,6-tetrahydropyridin-4-yl)phthalazin-1-amine;

1-{4-[6,7-dichloro-4-(cyclopentylamino)phthalazin-1-yl]-3,6-dihydropyridin-1(2H)-yl}ethanone;

2-[{5-[6,7-dichloro-4-(cyclopentylamino)phthalazin-1-yl]pyridin-2-yl}(methyl)amino]ethanol;

6,7-dichloro-N-cyclopentyl-4-[6-(piperazin-1-yl)pyridin-3-yl]phthalazin-1-amine;

1-{5-[6,7-dichloro-4-(cyclopentylamino)phthalazin-1-yl]pyridin-2-yl}piperidin-4-ol;

1-{4-[6,7-dichloro-4-(cyclopentylamino)phthalazin-1-yl]pyridin-2-yl}piperidin-4-ol;

2-({5-[6,7-dichloro-4-(cyclopentylamino)phthalazin-1-yl]pyridin-2-yl}amino)ethanol;

6,7-dichloro-N-cyclopentyl-4-[6-(pyrrolidin-1-yl)pyridin-3-yl]phthalazin-1-amine;

6,7-dichloro-N-cyclopentyl-4-[2-(dimethylamino)pyridin-4-yl]phthalazin-1-amine;

4-[6-(3-aminopyrrolidin-1-yl)pyridin-3-yl]-6,7-dichloro-N-cyclopentylphthalazin-1-amine;

1-{5-[6,7-dichloro-4-(cyclopentylamino)phthalazin-1-yl]pyridin-2-yl}pyrrolidin-3-ol;

2-[{4-[6,7-dichloro-4-(cyclopentylamino)phthalazin-1-yl]pyridin-2-yl}(methyl)amino]ethanol;

1-{4-[6,7-dichloro-4-(cyclopentylamino)phthalazin-1-yl]pyridin-2-yl}pyrrolidin-3-ol;

methyl 6,7-dichloro-4-(cyclopentylamino)phthalazine-1-carboxylate;

6,7-dichloro-4-(cyclopentylamino)phthalazine-1-carboxylic acid;

2-({4-[6,7-dichloro-4-(cyclopentylamino)phthalazin-1-yl]pyridin-2-yl}amino)ethanol;

4-[2-(3-aminopyrrolidin-1-yl)pyridin-4-yl]-6,7-dichloro-N-cyclopentylphthalazin-1-amine;

6,7-dichloro-N-cyclopentyl-4-[6-(1,1-dioxidothiomorpholin-4-yl)pyridin-3-yl]phthalazin-1-amine;

6,7-dichloro-4-(cyclopentylamino)phthalazine-1-carboxamide;

tert-butyl {4-[6,7-dichloro-4-(cyclopentylamino)phthalazin-1-yl]-1H-pyrazol-1-yl}acetate;

{4-[6,7-dichloro-4-(cyclopentylamino)phthalazin-1-yl]-1H-pyrazol-1-yl}acetic acid;

6,7-dichloro-4-(cyclopentylamino)-N-ethylphthalazine-1-carboxamide;

6,7-dichloro-4-(cyclopentylamino)-N-(2-hydroxyethyl)-N-methylphthalazine-1-carboxamide;

[6,7-dichloro-4-(cyclopentylamino)phthalazin-1-yl](pyrrolidin-1-yl)methanone;

[6,7-dichloro-4-(cyclopentylamino)phthalazin-1-yl](3-hydroxypyrrolidin-1-yl)methanone;

[6,7-dichloro-4-(cyclopentylamino)phthalazin-1-yl](4-methylpiperazin-1-yl)methanone;

[6,7-dichloro-4-(cyclopentylamino)phthalazin-1-yl](1,1-dioxidothiomorpholin-4-yl)methanone;

[6,7-dichloro-4-(cyclopentylamino)phthalazin-1-yl](morpholin-4-yl)methanone;

[6,7-dichloro-4-(cyclopentylamino)phthalazin-1-yl](4-hydroxypiperidin-1-yl)methanone;

6,7-dichloro-4-(cyclopentylamino)-N-phenylphthalazine-1-carboxamide;

6,7-dichloro-4-(cyclopentylamino)-N-(1-methyl-1H-pyrazol-4-yl)phthalazine-1-carboxamide;

2-{4-[6,7-dichloro-4-(cyclopentylamino)phthalazin-1-yl]-1H-pyrazol-1-yl}-N-ethylacetamide;

2-{4-[6,7-dichloro-4-(cyclopentylamino)phthalazin-1-yl]-1H-pyrazol-1-yl}-N-(2-hydroxyethyl)acetamide;

2-{4-[6,7-dichloro-4-(cyclopentylamino)phthalazin-1-yl]-1H-pyrazol-1-yl}-N-(2-hydroxyethyl)-N-methylacetamide;

2-{4-[6,7-dichloro-4-(cyclopentylamino)phthalazin-1-yl]-1H-pyrazol-1-yl}-1-(pyrrolidin-1-yl)ethanone;

2-{4-[6,7-dichloro-4-(cyclopentylamino)phthalazin-1-yl]-1H-pyrazol-1-yl}-1-(3-hydroxypyrrolidin-1-yl)ethanone;

2-{4-[6,7-dichloro-4-(cyclopentylamino)phthalazin-1-yl]-1H-pyrazol-1-yl}-1-(1,3-oxazolidin-3-yl)ethanone;

2-{4-[6,7-dichloro-4-(cyclopentylamino)phthalazin-1-yl]-1H-pyrazol-1-yl}-1-(4-methylpiperazin-1-yl)ethanone;

1-(4-acetylpiperazin-1-yl)-2-{4-[6,7-dichloro-4-(cyclopentylamino)phthalazin-1-yl]-1H-pyrazol-1-yl}ethanone;

2-{4-[6,7-dichloro-4-(cyclopentylamino)phthalazin-1-yl]-1H-pyrazol-1-yl}-1-(1,1-dioxidothiomorpholin-4-yl)ethanone;

2-{4-[6,7-dichloro-4-(cyclopentylamino)phthalazin-1-yl]-1H-pyrazol-1-yl}-1-(4-hydroxypiperidin-1-yl)ethanone;

2-{4-[6,7-dichloro-4-(cyclopentylamino)phthalazin-1-yl]-1H-pyrazol-1-yl}-1-(morpholin-4-yl)ethanone;

6,7-dichloro-4-(cyclopentylamino)-N-(2-hydroxyethyl)phthalazine-1-carboxamide;

6,7-dichloro-N-cyclopentyl-4-[1-(methylsulfonyl)-1,2,3,6-tetrahydropyridin-4-yl]phthalazin-1-amine;

6,7-dichloro-N-cyclopentyl-4-[1-(cyclopropylsulfonyl)-1,2,3,6-tetrahydropyridin-4-yl]phthalazin-1-amine;

6,7-dichloro-N-cyclopentyl-4-[1-(phenylsulfonyl)-1,2,3,6-tetrahydropyridin-4-yl]phthalazin-1-amine;

cyclopropyl{4-[6,7-dichloro-4-(cyclopentylamino)phthalazin-1-yl]-3,6-dihydropyridin-1(2H)-yl}methanone;

1-{4-[6,7-dichloro-4-(cyclopentylamino)phthalazin-1-yl]-3,6-dihydropyridin-1(2H)-yl}-2-hydroxypropan-1-one;

{4-[6,7-dichloro-4-(cyclopentylamino)phthalazin-1-yl]-3,6-dihydropyridin-1(2H)-yl}(tetrahydrofuran-3-yl)methanone;

3-{4-[6,7-dichloro-4-(cyclopentylamino)phthalazin-1-yl]-3,6-dihydropyridin-1(2H)-yl}-3-oxopropanenitrile;

{4-[6,7-dichloro-4-(cyclopentylamino)phthalazin-1-yl]-3,6-dihydropyridin-1(2H)-yl}(phenyl)methanone;

{4-[6,7-dichloro-4-(cyclopentylamino)phthalazin-1-yl]-3,6-dihydropyridin-1(2H)-yl}(2-methylphenyl)methanone;

{4-[6,7-dichloro-4-(cyclopentylamino)phthalazin-1-yl]-3,6-dihydropyridin-1(2H)-yl}(2,6-dimethylphenyl)methanone;

{4-[6,7-dichloro-4-(cyclopentylamino)phthalazin-1-yl]-3,6-dihydropyridin-1(2H)-yl}(pyridin-3-yl)methanone;

{4-[6,7-dichloro-4-(cyclopentylamino)phthalazin-1-yl]-3,6-dihydropyridin-1(2H)-yl}(1-methyl-1H-pyrazol-4-yl)methanone;

{4-[6,7-dichloro-4-(cyclopentylamino)phthalazin-1-yl]-3,6-dihydropyridin-1(2H)-yl}(1-methylpyrrolidin-3-yl)methanone;

1-{4-[6,7-dichloro-4-(cyclopentylamino)phthalazin-1-yl]-3,6-dihydropyridin-1(2H)-yl}-2-methoxyethanone;

3-({4-[6,7-dichloro-4-(cyclopentylamino)phthalazin-1-yl]-3,6-dihydropyridin-1(2H)-yl}carbonyl)cyclopentanone;

1-{4-[6,7-dichloro-4-(cyclopentylamino)phthalazin-1-yl]-3,6-dihydropyridin-1(2H)-yl}-2-(methylsulfonyl)ethanone;

3 -{4-[6,7-dichloro-4-(cyclopentylamino)phthalazin-1-yl]-3,6-dihydropyridin-1(2H)-yl}-3-oxopropanamide; and

{4-[6,7-dichloro-4-(cyclopentylamino)phthalazin-1-yl]-3,6-dihydropyridin-1(2H)-yl}(3-hydroxycyclopentyl)methanone.

Compound names are assigned by using Name 2012 naming algorithm by Advanced Chemical Development or Struct=Name naming algorithm as part of CHEMDRAW® ULTRA v. 12.0.2.1076.

Compounds of the invention may exist as stereoisomers wherein asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The invention contemplates various stereoisomers and mixtures thereof and these are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

Compounds of the invention may exist as cis or trans isomers, wherein substituents on a ring may attached in such a manner that they are on the same side of the ring (cis) relative to each other, or on opposite sides of the ring relative to each other (trans). For example, cyclobutane may be present in the cis or trans configuration, and may be present as a single isomer or a mixture of the cis and trans isomers. Individual cis or trans isomers of compounds of the invention may be prepared synthetically from commercially available starting materials using selective organic transformations, or prepared in single isomeric form by purification of mixtures of the cis and trans isomers. Such methods are well-known to those of ordinary skill in the art, and may include separation of isomers by recrystallization or chromatography.

It should be understood that the compounds of the invention may possess tautomeric forms, as well as geometric isomers, and that these also constitute an aspect of the invention.

The present disclosure includes all pharmaceutically acceptable isotopically-labelled compounds of formula (I) wherein one or more atoms are replaced by atoms having the same atomic number, but an atomic mass or mass number different from the atomic mass or mass number which predominates in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the accompanying Examples using an appropriate isotopically-labeled reagents in place of the non-labeled reagent previously employed.

Thus, the formula drawings within this specification can represent only one of the possible tautomeric, geometric, or stereoisomeric forms. It is to be understood that the invention encompasses any tautomeric, geometric, or stereoisomeric form, and mixtures thereof, and is not to be limited merely to any one tautomeric, geometric, or stereoisomeric form utilized within the formula drawings.

Compounds of formula (I) may be used in the form of pharmaceutically acceptable salts. The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio.

Pharmaceutically acceptable salts have been described in S. M. Berge et al. J. Pharmaceutical Sciences, 1977, 66: 1-19.

Compounds of formula (I) may contain either a basic or an acidic functionality, or both, and can be converted to a pharmaceutically acceptable salt, when desired, by using a suitable acid or base. The salts may be prepared in situ during the final isolation and purification of the compounds of the invention.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts may be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other examples of organic amines useful for the formation of base addition salts include ethylenediamine, ethanolamine, diethanolamine, piperidine, piperazine and the like.

The term “pharmaceutically acceptable prodrug” or “prodrug”as used herein, represents those prodrugs of the compounds of the invention which are, within the scope of sound medical judgement, suitable for use in contact with the tissues of humans and lower animals without undue toxicity, irritation, allergic response, and the like, commensurate with a reasonable benefit/risk ratio, and effective for their intended use.

The invention contemplates compounds of formula (I) formed by synthetic means or formed by in vivo biotransformation of a prodrug.

Compounds described herein can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

### General Synthesis

The compounds described herein in various embodiments, including compounds of general formula (I) and specific examples can be prepared by methodologies known in the art, for example, through the reaction schemes depicted in schemes 1 and 2. The variables R¹, R², R³, R⁴, R⁵, R⁶, G¹, G², R^{B}, and m, used in the following schemes have the meanings as set forth in the summary and detailed description sections, unless otherwise noted.

Abbreviations used in the descriptions of the schemes and the specific examples have the following meanings: DMF for N,N-dimethylformamide, dppf for 1,1′-bis(diphenylphosphino)ferrocene, DMSO for dimethyl sulfoxide, HOBT for 1-hydroxybenzotriazole hydrate, HATU for O-(7-azabenzotriazol-1-yl)-N,N,N′,N′-tetramethyluronium hexafluorophosphate, HOBT for 1-hydroxybenzotriazole hydrate, HPLC for High Performance Liquid chromatography, LCMS for liquid chromatography mass spectrometry, and NMR for nuclear magnetic resonance.

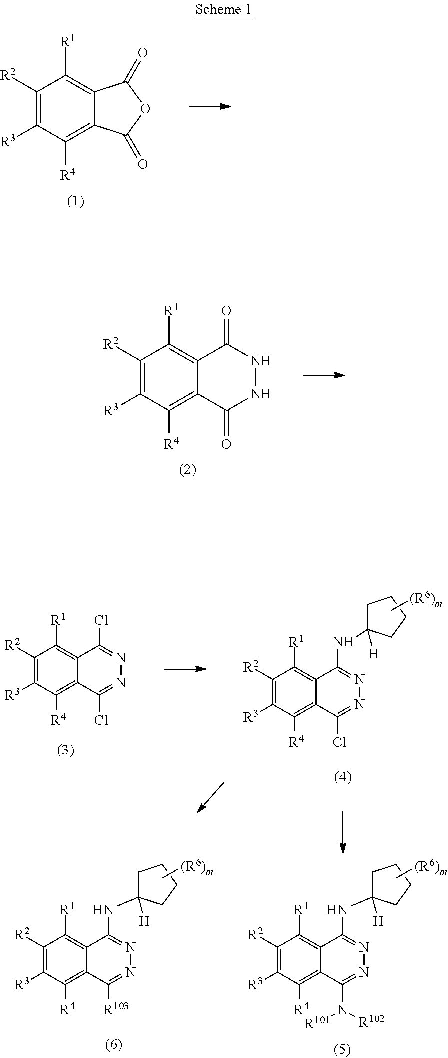

Compounds of general formula (I) wherein R⁵ is aryl, heteroaryl, or heterocycle, may be prepared utilizing general procedure as described in Scheme 1.

Treatment of diones of formula (1) with hydrazine in the presence of acetic acid at elevated temperature (for example, at about 70° C.) provides dihydrophthalazine diones of formula (2). Diones of formula (2) may be converted to chloro phthalazines of formula (3) by treating (2) with phosphorous oxychloride at elevated temperature. Displacement of the chloride with an optionally substituted cyclopentyl amine in the presence of a base such as, but not limited to, diisopropylethylamine, and at elevated temperature provides phthalazines of formula (4). Compounds of general formula (5) wherein R¹⁰¹ and R¹⁰², together with the nitrogen atom they are attached to form a heterocycle ring as described by G¹ in general formula (I) may be obtained from the displacement of the second chloro atom of formula (4) with an appropriate heterocyclic amines.

The chlorides of formula (4) may be treated with an appropriate boronic acids or derivatives thereof (e.g. boronic esters), in the presence of a palladium catalyst and a base, and optionally in the presence of a ligand, and in a suitable solvent at elevated temperature (for example, at a temperature ranging from about 80° C. to about 150° C.), to provide compounds of formula (6) wherein R¹⁰³ is aryl, heteroaryl, or heterocycle as described by G¹ in general formula (I). The reaction may be facilitated by microwave irradiation. Examples of the palladium catalyst include, but are not limited to, tetrakis(triphenylphosphine)palladium(0), and bis(triphenylphosphine)palladium dichloride. Examples of suitable bases that may be employed include, but are not limited to, carbonates or phosphates of sodium, potassium, and cesium; and cesium fluoride. Examples of suitable ligands include, but are not limited to, tricyclohexylphosphine, 1,3,5,7-tetramethyl-6-phenyl-2,4,8-trioxa-6-phosphaadamantane, 2-dicyclohexylphosphino-2′,4′,6′-triisopropylbiphenyl (X-phos), and 1,1′-bis(diphenylphosphanyl) ferrocene. Non-limiting examples of suitable solvent include methanol, n-butanol, dimethoxyethane, N,N-dimethylformamide, dimethylsulfoxide, dioxane, tetrahydropyran, and water, or a mixture thereof.

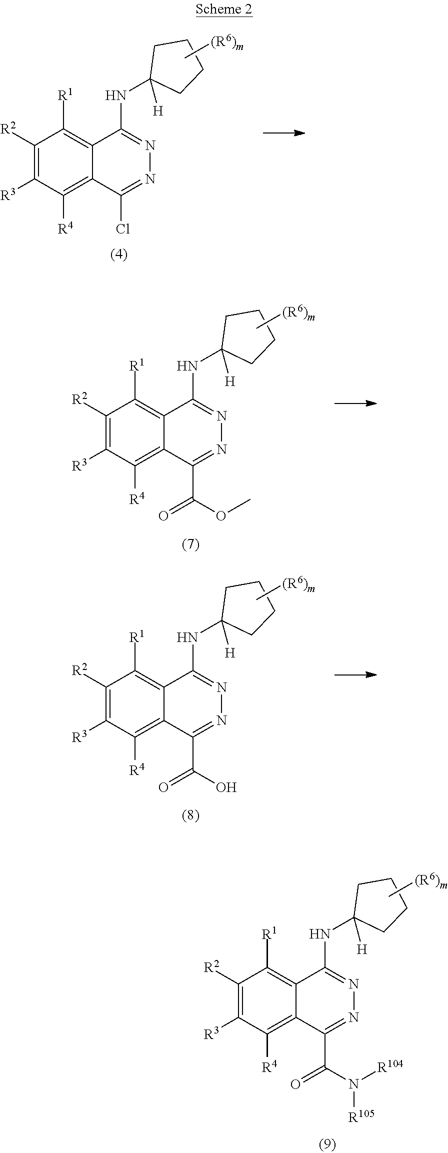

Treatment of compounds of general formula (4) with carbon monoxide in the presence of a palladium catalyst such as, but not limited to, PdCl₂(dppf) provides esters of formula (7). Hydrolysis of the esters (7) affords the carboxylic acids (8) which may be converted to amides of formula (9) wherein R¹⁰⁴ is hydrogen or C₁-C₆ alkyl, and R¹⁰⁵ is as described by R^{B} in general formula (I), or R¹⁰⁴ and R¹⁰⁵ together with the nitrogen atom form a monocyclic heterocycle ring as described by G² in general formula (I), by coupling with an appropriate amine using methodologies known to one skilled in the art. For example, the coupling reaction may be conducted in the presence of a coupling reagent such as, but not limited to, HATU or HOBT, and a base such as, but not limited to, diisopropylethyl amine, in a solvent such as, but not limited to, N,N-dimethylformamide, and at ambient temperature.

Optimal reaction conditions and reaction times for each individual step may vary depending on the particular reactants employed and substituents present in the reactants used. Unless otherwise specified, solvents, temperatures and other reaction conditions may be readily selected by one of ordinary skill in the art. Specific procedures are provided in the Synthetic Examples section. Reactions may be further processed in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or may be prepared by one skilled in the art from commercially available materials using methods described in the chemical literature.

Routine experimentations, including appropriate manipulation of the reaction conditions, reagents and sequence of the synthetic route, protection of any chemical functionality that are not compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in T. Greene and P. Wuts, Protecting Groups in Organic Synthesis (3^{rd} ed.), John Wiley & Sons, NY (1999), which is incorporated herein by reference in its entirety. Synthesis of the compounds of the invention may be accomplished by methods analogous to those described in the synthetic schemes described hereinabove and in specific examples.

Starting materials, if not commercially available, may be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound is required, it may be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it may be prepared by carrying out one of the above procedures using a pure geometric isomer as a starting material, or by resolution of a mixture of the geometric isomers of the compound or intermediates using a standard procedure such as chromatographic separation.

### Pharmaceutical Compositions

This invention also provides for pharmaceutical compositions comprising a therapeutically effective amount of a compound of formula (I), or a pharmaceutically acceptable salt thereof together with a pharmaceutically acceptable carrier, diluent, or excipient thereof. The phrase “pharmaceutical composition” refers to a composition suitable for administration in medical or veterinary use.

The pharmaceutical compositions that comprise a compound of formula (I), alone or in combination with a second therapeutic agent, may be administered to the subjects orally, rectally, parenterally, intracisternally, intravaginally, intraperitoneally, topically (as by powders, ointments or drops), bucally or as an oral or nasal spray. The term “parenterally” as used herein, refers to modes of administration which include intravenous, intramuscular, intraperitoneal, intrasternal, subcutaneous and intraarticular injection and infusion.

The term “pharmaceutically acceptable carrier” as used herein, means a non-toxic, inert solid, semi-solid or liquid filler, diluent, encapsulating material or formulation auxiliary of any type. Some examples of materials which may serve as pharmaceutically acceptable carriers are sugars such as, but not limited to, lactose, glucose, and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants may also be present in the composition, according to the judgment of the formulator.

Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate), and suitable mixtures thereof. Proper fluidity may be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally-administered drug form may be accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release may be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations may be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In certain embodiments, solid dosage forms may contain from 1% to 95% (w/w) of a compound of formula (I). In certain embodiments, the compound of formula (I) may be present in the solid dosage form in a range of from 5% to 70% (w/w). In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

The pharmaceutical composition may be a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form may be a capsule, tablet, cachet, or lozenge itself, or it may be the appropriate number of any of these in packaged form. The quantity of active component in a unit dose preparation may be varied or adjusted from 0.1 mg to 1000 mg, from 1 mg to 100 mg, or from 1% to 95% (w/w) of a unit dose, according to the particular application and the potency of the active component. The composition may, if desired, also contain other compatible therapeutic agents.

The dose to be administered to a subject may be determined by the efficacy of the particular compound employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician may evaluate factors such as the circulating plasma levels of the compound, compound toxicities, and/or the progression of the disease, etc.

For administration, compounds may be administered at a rate determined by factors that may include, but are not limited to, the LD₅₀ of the compound, the pharmacokinetic profile of the compound, contraindicated drugs, and the side-effects of the compound at various concentrations, as applied to the mass and overall health of the subject. Administration may be accomplished via single or divided doses.

The compounds utilized in the pharmaceutical method of the invention may be administered at the initial dosage of about 0.001 mg/kg to about 100 mg/kg daily. In certain embodiments, the daily dose range is from about 0.1 mg/kg to about 10 mg/kg. The dosages, however, may be varied depending upon the requirements of the subject, the severity of the condition being treated, and the compound being employed. Determination of the proper dosage for a particular situation is within the skill of the practitioner. Treatment may be initiated with smaller dosages, which are less than the optimum dose of the compound. Thereafter, the dosage is increased by small increments until the optimum effect under circumstances is reached. For convenience, the total daily dosage may be divided and administered in portions during the day, if desired.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds may also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols, and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which may be prepared by mixing the compounds with suitable non-irritating carriers or carriers such as cocoa butter, polyethylene glycol, or a suppository wax which are solid at room temperature but liquid at body temperature and therefore melt in the rectum or vaginal cavity and release the active compound.

Compounds may also be administered in the form of liposomes. Liposomes generally may be derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes may be used. The present compositions in liposome form may contain, in addition to a compound of the invention, stabilizers, preservatives, excipients, and the like. Examples of lipids include, but are not limited to, natural and synthetic phospholipids, and phosphatidyl cholines (lecithins), used separately or together.

Methods to form liposomes have been described, see example, Prescott, Ed., Methods in Cell Biology, Volume XIV, Academic Press, New York, N.Y. (1976), p. 33 et seq.

Dosage forms for topical administration of a compound described herein include powders, sprays, ointments, and inhalants. The active compound may be mixed under sterile conditions with a pharmaceutically acceptable carrier and any needed preservatives, buffers or propellants which may be required. Opthalmic formulations, eye ointments, powders and solutions are also contemplated as being within the scope of this invention.

### Methods of Use

The compounds and compositions using any amount and any route of administration may be administered to a subject for the treatment of treating a condition, disease, or disorder implicated by SUV420H1 activity. In certain embodiments, the invention provides a method of treating a condition, disease, or disorder implicated by SUV420H1 activity, the method comprising administering a therapeutically acceptable amount of a compound of formula (I) or a pharmaceutically acceptable salt thereof to a subject in need thereof. In certain embodiments, said subject is a human. In certain embodiments, the compound or salt thereof is administered with a pharmaceutically acceptable carrier.

The term “administering” refers to the method of contacting a compound with a subject. Thus, the compounds may be administered by injection, that is, intravenously, intramuscularly, intracutaneously, subcutaneously, intraduodenally, parentally, or intraperitoneally. Also, the compounds described herein may be administered by inhalation, for example, intranasally. Additionally, the compounds may be administered transdermally, topically, via implantation, transdermally, topically, and via implantation. In certain embodiments, the compounds and compositions thereof may be delivered orally. The compounds may also be delivered rectally, bucally, intravaginally, ocularly, or by insufflation. SUV420H1-modulated disorders and conditions may be treated prophylactically, acutely, and chronically using compounds and compositions thereof, depending on the nature of the disorder or condition. Typically, the host or subject in each of these methods is human, although other mammals may also benefit from the administration of compounds and compositions thereof as set forth hereinabove.

Compounds of the invention are useful as modulators of SUV420H1. Thus, the compounds and compositions are particularly useful for treating or lessening the severity, or progression of a disease, disorder, or a condition where hyperactivity or inactivity of SUV420H1 is involved. Accordingly, the invention provides a method for modulating SUV420H1 in a membrane of a cell, comprising the step of contacting said cell with a compound of formula (I) or a preferred embodiment thereof as set forth above. One embodiment is directed to a method of treating a condition, disease, or disorder in a subject implicated by SUV420H1 activity, comprising the step of administering to said subject a therapeutically effective amount of a compound of formula (I) or a preferred embodiment thereof as set forth above, or a pharmaceutically acceptable salt thereof. In certain embodiments, the subject is a mammal. In certain embodiments, the subject is a human.

One embodiment is directed to a compound according to formula (I) or a pharmaceutically acceptable salt thereof for use in medicine.

One embodiment is directed to a compound according to formula (I) or a pharmaceutically acceptable salt thereof for use in the treatment of condition, disease, or disorder in a subject implicated by SUV420H1 activity.

One embodiment is directed to the use of a compound according to formula (I) or a pharmaceutically acceptable salt thereof in the preparation of a medicament.

One embodiment is directed to the use of a compound according to formula (I) in the preparation of a medicament for use in the treatment of condition, disease, or disorder in a subject implicated by SUV420H1 activity.

The present compounds may be co-administered to a subject. The term “co-administered” means the administration of two or more different therapeutic agents that are administered to a subject by combination in the same pharmaceutical composition or separate pharmaceutical compositions. Thus co-administration involves administration at the same time of a single pharmaceutical composition comprising two or more therapeutic agents or administration of two or more different compositions to the same subject at the same or different times.

The compounds of the invention may be co-administered with a therapeutically effective amount of one or more agents to treat condition, disease, or disorder in a subject implicated by SUV420H1 activity.

This invention also is directed to methods of use of the compounds, salts, compositions, and/or kits of the invention to, for example, modulate SUV420H1 or fragment thereof, and treat a disease treatable by modulating SUV420H1 or a fragment thereof.

This invention also is directed to a use of one or more compounds and/or salts of the invention in the preparation of a medicament. The medicament optionally can comprise one or more additional therapeutic agents. In some embodiments, the medicament is useful for treating a disease, condition, or disorder implicated by SUV420H1 activity.

This invention also is directed to a use of one or more compounds and/or salts of the invention in the manufacture of a medicament for the treatment of a disease, condition, or disorder implicated by SUV420H1 activity. The medicament optionally can comprise one or more additional therapeutic agents.

Another aspect of the invention relates to modulating SUV420H1 activity in a biological sample or a patient (e.g., in vitro or in vivo), which method comprises administering to the patient, or contacting said biological sample with a compound of formula (I) or a composition comprising said compound. The term “biological sample”, as use herein, includes, without limitation, cell cultures or extracts thereof, biopsied material obtained from a mammal or extracts thereof, and blood, saliva, urine, feces, semen, tears, or other body fluids of extracts thereof.

Modulation of SUV420H1 activity in a biological sample is useful for a variety of purposes that are known to one of skill in the art. Examples of such purposes include, but are not limited to, the study of SUV420H1 activity in biological and pathological phenomena; and the comparative evaluation of new modulators of SUV420H1.

Further benefits of Applicants' invention will be apparent to one skilled in the art from reading this patent application.

The following Examples may be used for illustrative purposes and should not be deemed to narrow the scope of the invention.

### EXAMPLES

¹H NMR spectra were recorded on Bruker AV III 400. LCMS measurement was run on Agilent 1200 HPLC/6100 SQ System using the follow condition:

### Method A:

The gradient was started with 5% B for 0.1 minutes, and increased to 95% B within 0.7 minutes, held at 95% B for 0.9 minutes and finally dropped back to 5% B within 0.01 minutes (3.0 mL/min flow rate).

Mobile phase A: water with 0.05% trifluoroacetic acid; mobile phase B: acetonitrile with 0.05% trifluoroacetic acid; column: a 4.6×30 mm Zorbax SB-C₁₈ Rapid Resolution HT column (1.8 μm particles). Detection methods were diode array (DAD) and evaporative light scattering (ELSD) detection with positive/negative electrospray ionization.

### Method B:

The gradient was started with 5% B for 0.2 minutes, and increased to 95% B within 1.7 minutes, held at 95% B for 1.3 minutes, and finally dropped back to 5% B within 0.01 minutes (2.3 mL/min flow rate).

Mobile phase A: water with 0.05% trifluoroacetic acid; mobile phase B: acetonitrile with 0.05% trifluoroacetic acid; column: 4.6×50 mm)(Bridge C₁₈ column (3.5 μm particles). Detection methods were diode array (DAD) and evaporative light scattering (ELSD) detection with positive/negative electrospray ionization.

### Method C:

The gradient was started with 5% B for 0.2 minutes, and increased to 95% B within 1.7 minutes, held at 95% B for 1.3.0 minutes, and finally dropped back to 5% B within 0.01 minutes (2.3 mL/min flow rate).

Mobile phase A: water with 10 mM NH₄HCO₃ and mobile phase B: HPLC grade acetonitrile. The column used for the chromatography was a 4.6×50 mm XBridge C18 column (3.5 μm particles). Detection methods were diode array (DAD) and evaporative light scattering (ELSD) detection with positive/negative electrospray ionization.

### Example 1

### 4,6,7-trichloro-N-cyclopentylphthalazin-1-amine

### Example 1a

### 6,7-dichloro-2,3-dihydrophthalazine-1,4-dione

To a solution of 5,6-dichloroisobenzofuran-1, 3-dione (8.8 g, 40.6 mmol) in acetic acid (40 mL), hydrazine (1.886 ml, 48.7 mmol) was added carefully at room temperature. The mixture was stirred at 70° C. overnight under nitrogen. The reaction mixture was cooled and the solid material was collected to give the title compound, which was used without further purification (8.9 g, 90%). LCMS (Method B): m/z 231.7 (M+H), retention time: 1.38 minutes. ¹H NMR (DMSO-d₆, 400 MHz) δ 11.88 (s, 2H), 8.18 (s, 2H).

### Example 1b

### 1,4,6,7-tetrachlorophthalazine

To a mixture of Example 1a (4.0 g, 17.31 mmol) in POCl₃ (16.14 mL, 173 mmol) in a 250 mL round-bottomed flask diisopropylethylamine (3.02 mL, 17.31 mmol) was added dropwise. The mixture was heated at 130° C. overnight. After cooling to room temperature, the mixture was slowly poured into ice-water (500 mL) and stirred for 1 hour. The solid was collected by filtration and re-dissolved in dichloromethane (200 mL). The organic solution was washed with brine (3×100 mL), dried with anhydrous sodium sulfate, and filtered. The filtrate was concentrated under reduced pressure to give the title compound (4.0 g, yield: 70%). LCMS (Method B): m/z 266.8 (M+H), retention time: 1.91 minutes.

### Example 1c

### 4,6,7-trichloro-N-cyclopentylphthalazin-1-amine

A mixture of Example 1b (5.5 g, 20.53 mmol) and diisopropylethylamine (3.4 g, 26.67 mmol) in dimethylsulfoxide (50 mL) in a 250 mL round-bottomed flask was heated to 80° C., and then a solution of cyclopentylamine (1.748 g, 20.53 mmol) in dimethylsulfoxide (4 mL) was added drop wise. The mixture was stirred at 80° C. for about 2 hours and then at room temperature overnight. The solid material was collected by filtration and then taken into dichloromethane (60 mL). The organic solution was washed with brine (50 mL×2), dried over anhydrous sodium sulfate, filtered, and concentrated under reduced pressure to give the title compound, which was used without further purification. LCMS (Method C): m/z 316.0 (M+H), retention time: 2.26 minutes; ¹H NMR (CDCl₃, 400 MHz) δ 8.25(s, 1H), 7.85 (s, 1H), 5.03 (d, J=5.6 Hz, 1H), 4.64-4.59 (m, 1H), 2.28-2.21 (m, 2H), 1.80-1.59 (m, 4H), 1.56-1.52 (m, 2H).

### Example 2

### 6,7-dichloro-N-cyclopentyl-4-phenylphthalazin-1-amine

A mixture of Example 1 (90 mg, 0.284 mmol), phenylboronic acid (34.7 mg, 0.284 mmol), tetrakis(triphenylphosphine)palladium(0) (32.8 mg, 0.028 mmol) and K₂CO₃ (118 mg, 0.853 mmol) in 1,4-dioxane/water (ratio: 4:1, Volume: 6 mL) was stirred at 80° C. for 1 hour. The reaction was purified by reverse phase preparative HPLC (Gilson 281: column Xbridge 21.2*250 mm c18 with a gradient 25-55% mobile phase B in mobile phase A; mobile phase A: water with 10 mM NH₄HCO₃; mobile phase B: acetonitrile) to give the title compound. LCMS (Method C): m/z 358.0 (M+H), retention time: 2.31 minutes; ¹H NMR (CDCl₃, 400 MHz) δ 8.06 (s, 1H), 7.90 (s, 1H), 7.68-7.65 (m, 2H), 7.56-7.50 (m, 3H), 5.00 (d, J=6.4 Hz, 1H), 4.77-4.69 (m, 1H), 2.38-2.25(m, 2H), 1.84-1.61(m, 4H), 1.60-1.57(m, 2H).

### Example 3

### 6,7-dichloro-N-cyclopentyl-4-(pyridin-3-yl)phthalazin-1-amine

Example 3 was prepared according to the procedure used for the synthesis of Example 2, substituting pyridin-3-ylboronic acid for phenylboronic acid. LCMS (Method B): m/z 359.1 (M+H), retention time: 1.88 minutes; ¹H NMR (CDCl₃, 400 MHz) δ 8.93-8.92 (dd, J₁=0.4 Hz, J₂=2.0 Hz, 1H), 8.76-8.74 (dd, J₁=1.6 Hz, J₂=5.2 Hz, 1H), 8.06-8.03 (m, 1H), 8.00(s, 1H), 7.93 (s, 1H), 7.52-7.48 (m, 1H), 5.10 (d, J=6.4 Hz, 1H), 4.79-4.70 (m, 1H), 2.36-2.27 (m, 2H), 1.84-1.66 (m, 4H), 1.64-1.58 (m, 2H).

### Example 4

### 6,7-dichloro-N-cyclopentyl-4-(1-methyl-1H-pyrazol-3-yl)phthalazin-1-amine

Example 4 was prepared according to the procedure used for the synthesis of Example 2, substituting 1-methyl-3-(4,4,5,5-tetramethyl-1,3,2-dioxaborolan-2-yl)-1H-pyrazole for phenylboronic acid. LCMS (Method C): m/z 362.1 (M+H), retention time: 2.02 minutes; ¹H NMR (CDCl₃, 400 MHz) δ 9.57 (s, 1H), 7.84 (s, 1H), 7.44 (d, J=2.0 Hz, 1H), 7.11 (d, J=2.4 Hz, 1H), 4.99 (d, J=6.4 Hz, 1H), 4.76-4.68 (m, 1H), 4.04 (s, 3H), 2.36-2.26 (m, 2H), 1.82-1.63 (m, 4H), 1.62-1.57 (m, 2H).

### Example 5

### 6,7-dichloro-N-cyclopentyl-4-(1-methyl-1H-pyrazol-4-yl)phthalazin-1-amine

Example 5 was prepared according to the procedure used for the synthesis of Example 2, substituting 1-methyl-4-(4,4,5,5-tetramethyl-1,3,2-dioxaborolan-2-yl)-1H-pyrazole. LCMS (Method B): m/z 362.1 (M+H), retention time: 1.89 minutes; ¹H NMR (CDCl₃, 400 MHz) δ 8.28 (s, 1H), 7.91 (s, 1H), 7.88 (d, J=2.0 Hz, 2H), 4.94 (d, J=6.4 Hz, 1H), 4.73-4.65 (m, 1H), 4.03 (s, 3H), 2.33-2.25 (m, 2H), 1.83-1.63 (m, 4H), 1.62-1.57 (m, 2H).

### Example 6

### 6,7-dichloro-N-cyclopentyl-4-(pyridin-4-yl)phthalazin-1-amine

Example 6 was prepared according to the procedure used for the synthesis of Example 2, substituting pyridin-4-ylboronic acid for phenylboronic acid. LCMS (Method C): m/z 359.1 (M+H), retention time: 1.88 minutes; ¹H NMR (CDCl₃, 400 MHz) δ 8.81-8.79 (dd, J₁=1.6 Hz, J₂=4.4 Hz, 2H), 8.01 (s, 1H), 7.94 (s, 1H), 7.63-7.61 (dd, J, =1.6 Hz, J₂=4.4 Hz, 2H), 5.15 (d, J=6.4 Hz, 1H), 4.79-4.71 (m, 1H), 2.35-2.27 (m, 2H), 1.84-1.66 (m, 4H), 1.64-1.59 (m, 2H).

### Example 7

### 6,7-dichloro-N-cyclopentyl-4-(thiophen-3-yl)phthalazin-1-amine

Example 7 was prepared according to the procedure used for the synthesis of Example 2, substituting thiophen-3-ylboronic acid for phenylboronic acid. LCMS (Method C): m/z 364.0 (M+H), retention time: 2.29 minutes; ¹H NMR (CDCl₃, 400 MHz) δ 8.24 (s, 1H), 7.89 (s, 1H), 7.68-7.67 (m, 1H), 7.51-7.49 (m, 2H), 5.01 (d, J=6.4 Hz, 1H), 4.76-4.67 (m, 1H), 2.33-2.26 (m, 2H), 1.83-1.64 (m, 4H), 1.62-1.58 (m, 2H).

### Example 8

### 6,7-dichloro-N-cyclopentyl-4-(piperidin-1-yl)phthalazin-1-amine

A mixture of Example 1c (100 mg, 0.316 mmol) and piperidine (807 mg, 9.48 mmol) was stirred at 170° C. for 2 hours. The reaction was cooled to room temperature and diluted with dichloromethane (10 mL). The solution was washed with water (10 mL×2), dried over anhydrous magnesium sulfate, filtered through glass fiber paper, and concentrated. The residue was purified by reverse phase preparative HPLC (Gilson 281: column Xbridge 21.2*250 mm c18 with a gradient 25-55% mobile phase B in mobile phase A; mobile phase A: water with 10 mM NH₄HCO₃; mobile phase B: acetonitrile) to give the title compound. LCMS (Method B): m/z 365.2 (M+H), retention time: 2.32 minutes; ¹H NMR (CDCl₃, 400 MHz) δ 8.09 (s, 1H), 7.83(s, 1H), 4.60-4.53 (m, 1H), 3.23-3.21 (m, 4H), 2.28-2.20 (m, 2H), 1.83-1.80 (m, 4H), 1.79-1.64 (m, 6H), 1.60-1.52 (m, 2H).

### Example 9

### 6,7-dichloro-N-cyclopentyl-4-(4-methylpiperazin-1-yl)phthalazin-1-amine

Example 9 was prepared according to the procedure used for the synthesis of Example 8, substituting 1-methylpiperazine for piperidine. LCMS (Method C): m/z 380.1 (M+H), retention time: 2.01 minutes; ¹H NMR (CDCl₃, 400 MHz) δ: 8.08 (s, 1H), 7.80 (s, 1H), 4.69 (d, J=6.0 Hz, 1H), 4.62-4.54 (m, 1H), 3.36-3.34 (t, J=4.4 Hz, 4H), 2.71-2.69 (s, 4H), 2.41 (s, 3H), 2.28-2.20 (m, 2H), 1.83-1.80 (m, 4H), 1.58-1.57 (m, 2H).

### Example 10

### 6,7-dichloro-N-cyclopentyl-4-(morpholin-4-yl)phthalazin-1-amine

Example 10 was prepared according to the procedure used for the synthesis of Example 8, substituting morpholine for piperidine. LCMS (Method C): m/z 367.0 (M+H), retention time: 2.13 minutes; ¹H NMR (CDCl₁, 400 MHz) δ 7.75 (s, 1H), 7.58 (s, 1H), 4.91 (d, J=6.0 Hz, 1H), 4.62-4.58 (m, 1H), 3.94 (t, J=4.4 Hz, 4H), 3.23 (t, J=4.4 Hz, 4H), 2.26-2.22 (m, 2H), 1.80-1.64 (m, 4H), 1.57-1.52 (m, 2H).

### Example 11

### 6,7-dichloro-N-cyclopentyl-4-(pyrrolidin-1-yl)phthalazin-1-amine

Example 11 was prepared according to the procedure used for the synthesis of Example 8, substituting pyrrolidine for piperidine. LCMS (Method B): m/z 351.1 (M+H), retention time: 2.17 minutes; ¹H NMR (CDCl₃, 400 MHz) δ 7.61 (s, 1H), 7.20 (s, 1H), 4.76-4.74 (m, 1H), 4.63-4.57 (m, 1H), 3.62 (t, J=6.4 Hz, 4H), 2.27-2.21 (m, 2H), 2.05-2.01 (m, 4H), 1.79-1.68 (m, 4H), 1.55-1.50 (m, 2H).

### Example 12

### 6,7-dichloro-N-cyclopentyl-4-(2-fluoropyridin-4-yl)phthalazin-1-amine

A mixture of Example 1c (2 g, 6.32 mmol), (2-fluoropyridin-4-yl)boronic acid (1.068 g, 7.58 mmol), K₂CO₃ (1.746 g, 12.63 mmol) and tetrakis(triphenylphosphine)palladium(0) (0.730 g, 0.632 mmol) in 1,4-dioxane (50 mL) and water (5 mL) was stirred at 80° C. under nitrogen for 16 hours. Water (30 mL) was added. The mixture was extracted with ethyl acetate (150 mL×2). The combined organic phases were washed with brine, dried over magnesium sulfate, filtered, and concentrated under reduced pressure to give crude product, which was purified by column chromatography on silica gel using a gradient of petroleum ether/ethyl acetate from 5/1 to 2/1 to afford the title compound (0.35 g, 0.835 mmol, 13.22% yield). LCMS (Method B): m/z 377.1 (M+H), retention time: 2.04 minutes;¹H NMR (CDCl₃, 400 MHz) δ 8.33 (d, J=5.2 Hz, 1H), 7.93 (s, 1H), 7.88 (s, 1H), 7.45 (d, J=4.8 Hz, 1H), 7.21(s, 1H), 5.15 (d, J=6 Hz, 1H), 4.70-4.65 (m, 1H), 2.28-2.20 (m, 2H), 1.77-1.62 (m,6H).

### Example 13

### rac-(1R,3S)-3-[(4,6,7-trichlorophthalazin-1-yl)amino]cyclopentanol

A mixture of Example 1b (50 mg, 0.187 mmol), N-ethyl-N-isopropylpropan-2-amine (39.1 μL, 0.224 mmol) and rac-(1R,3S)-3-aminocyclopentanol (24.09 μL, 0.205 mmol) in 0.5 mL dimethylsulfoxide was heated at 120° C. in a microwave oven (Biotage Initiator) for 40 minutes. Water was added. The mixture was extracted with ethyl acetate (2×), washed with water (2×), brine, dried over magnesium sulfate, and filtered. The filtrate was concentrated and the residue was purified on silica gel column (0-8% methanol in dichloromethane) to give the title compound (22 mg, 0.066 mmol, 35.4% yield) as a white solid. ¹H NMR (400 MHz, DMSO-d₆) δ 8.84 (s, 1H), 8.15 (s, 1H), 7.65 (d, J =6.6 Hz, 1H), 4.67 (dd, J =15.1, 3.8 Hz, 1H), 4.49-4.29 (m, 1H), 4.19-3.97 (m, 1H), 2.39-2.22 (m, 1H), 2.09-1.90 (m, 1H), 1.86-1.46 (m, 4H); MS (ESI+) m/z 334.0 (M+H)⁺.

### Example 14

### 6,7-dichloro-N-cyclopentyl-4-[4-(morpholin-4-ylmethyl)phenyl]phthalazin-1-amine

A mixture of Example 1c (50 mg, 0.158 mmol), 4-(4-(4,4,5,5-tetramethyl-1,3,2-dioxaborolan-2-yl)benzyl)morpholine (47.9 mg, 0.158 mmol), bis(triphenylphosphine)palladium(II) dichloride (11.08 mg, 0.016 mmol) and sodium carbonate (50.2 mg, 0.474 mmol) in 3 mL dioxane and 1 mL water was purged with nitrogen and then heated at 120° C. in a microwave oven (Biotage Initiator) for 30 minutes. Water was added, extracted with ethyl acetate (2×), washed with brine, dried over magnesium sulfate, and filtered. The filtrate was concentrated and the residue was purified on silica gel column (50-100% ethyl acetate in heptanes) to give the title compound (27 mg, 0.059 mmol, 37.4% yield) as an off-white solid. 1H NMR (400 MHz, DMSO-d₆) δ 8.84 (s, 1H), 7.84 (s, 1H), 7.55 (d, J =8.0 Hz, 2H), 7.46 (d, J =8.1 Hz, 2H), 7.42 (d, J =6.5 Hz, 1H), 4.56 (dt, J =13.1, 6.5 Hz, 1H), 3.62-3.56 (m, 4H), 3.55 (s, 2H), 2.40 (s, 4H), 2.15-1.97 (m, 2H), 1.82-1.50 (m, 6H); MS (ESI+) m/z 457.2 (M+H)⁺.

### Example 15

### 6,7-dichloro-N-cyclopentyl-4-[2-(pyrrolidin-1-yl)pyridin-4-yl]phthalazin-1-amine

Example 15 was prepared using the procedure for the synthesis of Example 29, substituting pyrrolidine for piperidin-4-ol. LCMS (Method B): m/z 428.1 (M+H), retention time: 2.28 minutes; ¹H NMR (CDCl₃, 400 MHz) δ 8.22 (d, J=5.2 Hz, 1H), 8.05 (s, 1H), 7.82 (s, 1H), 6.68-6.62 (m, 2H), 5.99 (d, J=5.2 Hz, 1H), 4.69-4.63 (m, 1H), 3.46-3.40 (t, J=6.4 Hz, 4H), 2.28-2.22 (m, 2H), 1.98-1.94 (m, 4H), 1.76-1.57 (m, 6H).

### Example 16

### 6,7-dichloro-N-cyclopentyl-4-[2-(4-methylpiperazin-1-yl)pyridin-4-yl]phthalazin-1-amine

Example 16 was prepared using the procedure for the synthesis of Example 29, substituting 1-methylpiperazine for piperidin-4-ol. LCMS (Method B): m/z 457.2 (M+H), retention time: 2.02 minutes; ¹H NMR (CDCl₃, 400 MHz) δ 8.32 (d, J=5.2 Hz, 1H), 8.08 (s, 1H), 7.89 (s, 1H), 6.97 (s, 1H), 6.83 (d, J=5.2 Hz, 1H), 5.07 (d, J=6.4 Hz, 1H), 4.73-4.71 (m, 1H), 3.67-3.64 (m, 4H), 2.56-2.35 (m, 4H), 2.35 (s, 3H), 2.28-2.21 (m, 2H), 1.76-1.64 (m, 6H).

### Example 17

### 6,7-dichloro-N-cyclopentyl-4-[2-(morpholin-4-yl)pyridin-4-yl]phthalazin-1-amine

Example 17 was prepared using the procedure for the synthesis of Example 29, substituting morpholine for piperidin-4-ol. LCMS (Method B): m/z 444.2 (M+H), retention time: 2.06 minutes; ¹H NMR (CDCl₃, 400 MHz) δ 8.33 (d, J=5.2 Hz, 1H), 8.05 (s, 1H), 7.92 (s, 1H), 6.98 (s, 1H), 6.87 (d, J=5.2 Hz, 1H), 5.17 (s, 1H), 4.73-4.69 (m, 1H), 3.84-3.82 (t, J=4.8 Hz, 4H), 3.60-3.57 (t, J=5.2 Hz, 4H), 2.32-2.25 (m, 2H), 1.80-1.62 (m, 6H).

### Example 18

### 6,7-dichloro-N-cyclopentyl-4-[2-(piperazin-1-yl)pyridin-4-yl]phthalazin-1-amine

Example 18 was prepared using the procedure for the synthesis of Example 29, substituting piperazine for piperidin-4-ol. LCMS (Method B): m/z 443.2 (M+H), retention time: 1.87 minutes; ¹H NMR (CDCl₃, 400 MHz) δ 8.34 (d, J=4.8 Hz, 1H), 8.06 (s, 1H), 7.91(s, 1H), 7.04 (s, 1H), 6.90 (d, J=4.8 Hz, 1H), 5.09 (d, J=6.4 Hz, 1H), 4.76-4.72 (m, 1H), 3.77 (s, 4H), 3.15 (s, 4H), 2.30-2.28 (m,3H), 1.38-1.29 (m, 6H).

### Example 19

### 6,7-dichloro-N-cyclopentyl-4-(6-fluoropyridin-3-yl)phthalazin-1-amine

The mixture of Example 1c (2.0 g, 6.32 mmol), K₂CO₃ (2.62 g, 18.95 mmol) and (6-fluoropyridin-3-yl)boronic acid (1.06 g, 7.58 mmol) in N,N-dimethylformamide (20 mL) and water (1 mL) was degassed with argon for 10 minutes.

Tetrakis(triphenylphosphine)palladium(0) (0.73g, 0.63 mmol) was added. The resulting mixture was stirred at 80° C. for 6 hours under nitrogen. The reaction mixture was diluted with ethyl acetate (60 mL) and water (50 mL). The mixture was extracted with ethyl acetate (100 mL×2), and the combined organic phase was washed with brine, dried over magnesium sulfate, filtered, and concentrated under reduced pressure. The residue was purified by column chromatography on silica gel using a gradient of petroleum ether/ethyl acetate from 10/1 to 3/1 to give the titled compound (1.1 g, 2.92 mmol, 46.2% yield). LCMS (Method C): m/z 377.1 (M+1)⁺, retention time: 2.033 minutes; ¹H NMR (400 MHz, DMSO-d₆) δ 8.89 (s, 1H), 8.50 (d, J=2.4 Hz, 1H), 8.30-8.24 (m, 1H), 7.91 (s, 1H), 7.58 (d, J=6.4 Hz, 1H), 7.40-7.37 (dd, J₁=2.4 Hz, J₂=8.4 Hz, 1H), 4.61-4.58 (m, 1H), 2.09-2.06 (m, 2H), 1.79-1.59 (m, 6H).

### Example 20

### 6,7-dichloro-N-cyclopentyl-4-[6-(dimethylamino)pyridin-3-yl]phthalazin-1-amine

A mixture of example 19 (50 mg, 0.133 mmol), potassium carbonate (55 mg, 0.398 mmol) and dimethylamine hydrochloride (10.8 mg, 0.133 mmol) in N,N-dimethylformamide (1 mL) was stirred at 80° C. for 15 hours under nitrogen. The reaction mixture was directly purified by reverse phase preparative HPLC (Gilson 281: column Xbridge 21.2*250 mm c18 with a gradient 25-55% mobile phase B in mobile phase A; mobile phase A: water with 10 mM NH₄HCO₃; mobile phase B:acetonitrile) to give the title compound (30 mg, 0.042 mmol, 31.7% yield). LCMS (Method C): m/z 402.1 (M+H)⁺, retention time: 2.107 minutes; ¹H NMR (400 MHz, CDCl₃) δ 8.45 (d, J=2.0 Hz, 1H), 8.13 (s, 1H), 7.89 (s, 1H), 7.88-7.85 (m, 1H), 6.68 (d, J=8.8 Hz, 1H), 4.98 (d, J=6.4 Hz, 1H), 4.71-4.69 (m, 1H), 3.19 (s, 6H), 2.31-2.27 (m, 2H), 1.79-1.59 (m, 6H).

### Example 21

### 6,7-dichloro-N-cyclopentyl-4-[6-(4-methylpiperazin-1-yl)pyridin-3-yl]phthalazin-1-amine

A mixture of Example 19 (50 mg, 0.133 mmol), N-ethyl-N-isopropylpropan-2-amine (0.118 ml, 0.663 mmol), and 1-methylpiperazine (133 mg, 1.325 mmol) in 1 mL N,N-dimethylformamide was stirred at 100° C. for 15 hours under inert gas.. The reaction mixture was directly purified by reverse phase preparative HPLC (Gilson 281: column Xbridge 21.2*250 mm c18 with a gradient 25-55% mobile phase B in mobile phase A; mobile phase A: water with 10 mM NH₄HCO₃; mobile phase B:acetonitrile) to give the title compound (24.1 mg, 0.053 mmol, 39.8% yield). LCMS (Method C): m/z 457.2 (M+1)⁺, retention time: 2.022 minutes; ¹H NMR (400 MHz, CDCl₃) δ 8.46 (d, J=2.4 Hz, 1H), 8.10 (s, 1H), 7.92 (s, 1H), 7.88 (d, J₁=2.0 Hz, J₂=10.8 Hz, 1H), 6.81 (d, J=8.8 Hz, 1H), 5.07 (d, J=6.0 Hz, 1H), 4.72-4.69 (m, 1H), 3.69 (t, J=4.8 Hz, 4H), 2.57 (t, J=5.2 Hz, 4H), 2.38 (s, 1H), 2.34-2.26 (m, 2H), 1.81-1.58 (m, 6H).

### Example 22

### 6,7-dichloro-N-cyclopentyl-4-[6-(morpholin-4-yl)pyridin-3-yl]phthalazin-1-amine

Example 22 was prepared using the procedure for the synthesis of Example 21, substituting morpholine for 1-methylpiperazine. LCMS (Method C): m/z 444.1 (M+H)⁺, retention time: 2.055 minutes; ¹H NMR (400 MHz, CDCl₃) δ 8.48 (d, J=2.0 Hz, 1H), 8.10 (s, 1H), 7.93-7.89 (m, 2H), 6.80 (d, J=8.8 Hz, 1H), 5.02 (d, J=6.4 Hz, 1H), 4.72-4.70 (m, 1H), 3.87 (t, J₁=4.8 Hz, 4H), 3.63 (t, J=4.8 Hz, 4H), 2.31-2.27 (m, 2H), 1.79-1.59 (m, 6H).

### Example 23

### tert-butyl 4-[6,7-dichloro-4-(cyclopentylamino)phthalazin-1-yl]-3,6-dihydropyridine-1(2H)-carboxylate

A mixture of Example 1c (2.4 g, 7.58 mmol), tert-butyl 4-(4,4,5,5-tetramethyl-1,3,2-dioxaborolan-2-yl)-5,6-dihydropyridine-1(2H)-carboxylate (2.63 g, 8.49 mmol), tetrakis(triphenylphosphine)palladium(0) (0.876 g, 0.758 mmol) and potassium carbonate (2.095 g, 15.16 mmol) in N,N-dimethylformamide (24 mL) and water (2.4 mL) was stirred at 100° C. for 16 hours under nitrogen gas. The reaction mixture was poured into ice water (80 mL) and extracted with ethyl acetate (100 mL×3). The combined organic phase was washed with brine, dried over magnesium sulfate, filtered, and concentrated under reduced pressure. The residue was purified by column chromatography on silica gel (petroleum ether/ethyl acetate=2/1) to give the title compound (3g, 85%). LCMS (Method B): m/z 463.2 (M+H), retention time: 2.21 minutes.

### Example 24

### 6,7-dichloro-N-cyclopentyl-4-(1,2,3,6-tetrahydropyridin-4-yl)phthalazin-1-amine

To a solution of Example 23 (200 mg, 0.432 mmol) in dichloromethane (1 mL) was added trifluoroacetic acid (1 mL, 0.432 mmol). The reaction mixture was stirred at room temperature for 2 hours and then concentrated under reduced pressure. The residue was taken into dichloromethane (2 mL) and basified with saturated aqueous sodium bicarbonate. The mixture was extracted with dichloromethane (5 mL×2). The combined organic phase was washed with brine, dried over magnesium sulfate, filtered, and concentrated under reduced pressure to give the title compound (100 mg, 60%). LCMS (Method B): m/z 363.1 (M+H), retention time: 1.75 minutes. ¹H NMR (400 MHz, CD₃OD) δ 8.55 (s, 1H), 8.14 (s, 1H), 5.94 (s, 1H), 4.47-4.43 (m, 1H), 3.79 (d, J=2.4 Hz, 2H), 3.39 (t, J=6.0 Hz, 2H), 2.71 (d, J=2.0 Hz, 2H), 2.06-2.04 (m, 2H), 1.73-1.58 (m, 8H).

### Example 25

### 1-{4-[6,7-dichloro-4-(cyclopentylamino)phthalazin-1-yl]-3,6-dihydropyridin-1(2H)-yl}ethanone

A mixture of Example 1c (50 mg, 0.158 mmol), 1-(4-(4,4,5,5-tetramethyl-1,3,2-dioxaborolan-2-yl)-5,6-dihydropyridin-1(2H)-yl)ethanone (39.7 mg, 0.158 mmol), bis(triphenylphosphine)palladium(II) dichloride (11.08 mg, 0.016 mmol) and sodium carbonate (50.2 mg, 0.474 mmol) in dioxane (3 mL) and water (1 mL) was purged with nitrogen and then heated at 120° C. in a microwave oven (Biotage Initiator) for 30 minutes. Water was added. The mixture was extracted with ethyl acetate (2×), washed with brine, dried over magnesium sulfate, and filtered. The filtrate was concentrated and purified on silica gel column (0-8% methanol in dichloromethene) to give the title compound (24 mg, 0.059 mmol, 37.5% yield) as a light yellow solid. ¹H NMR (400 MHz, DMSO-d₆) δ 8.78 (s, 1H), 8.17 (d, J =20.1 Hz, 1H), 7.35 (d, J =6.4 Hz, 1H), 5.96 (s, 1H), 4.51 (dt, J =13.0, 6.7 Hz, 1H), 4.20 (dd, J=20.6, 2.6 Hz, 2H), 3.70 (dt, J=10.8, 5.6 Hz, 2H), 2.63 (s, 1H), 2.53 (s, 1H), 2.08 (d, J =3.1 Hz, 3H), 2.03 (dd, J =11.9, 4.2 Hz, 2H), 1.79-1.67 (m, 2H), 1.61 (dt, J =9.8, 5.5 Hz, 4H). MS (ESI+) m/z 405.1 (M+H)⁺.

### Example 26

### 2-{[5-[6,7-dichloro-4-(cyclopentylamino)phthalazin-1-yl]pyridin-2-yl}(methyl)amino]ethanol

Example 26 was prepared using the procedure for the synthesis of Example 21, substituting 2-(methylamino)ethanol for 1-methylpiperazine. LCMS (Method C): m/z 432.2 (M+H), retention time: 1.918 minutes; ¹H NMR (400 MHz, CDCl₃) δ 8.35 (d, J=1.6 Hz, 1H), 8.06 (s, 1H), 7.93 (s, 1H), 7.88-8.85 (dd, J₁=2.4 Hz, J₂=8.8 Hz, 1H), 6.71 (d, J=8.4 Hz, 1H), 5.10 (d, J=6.0 Hz, 1H), 4.90 (brs, 1H), 4.71-4.68 (m, 1H), 3.91-3.90 (t, J=4.8 Hz, 1H), 3.82 (t, J=4.8 Hz, 1H), 3.17 (s, 3H), 2.31-2.27 (m, 2H), 1.79-1.57 (m, 6H).

### Example 27

### 6,7-dichloro-N-cyclopentyl-4-[6-(piperazin-1-yl)pyridin-3-yl]phthalazin-1-amine

Example 27 was prepared using the procedure for the synthesis of Example 21, substituting piperazine for 1-methylpiperazine. LCMS (Method C): m/z 443.1 (M+H), retention time: 1.874 minutes; ¹H NMR (400 MHz, CDCl₃) δ 8.46 (d, J=2.0 Hz, 1H), 8.10 (s, 1H), 7.92 (s, 1H), 7.88 (d, J₁=2.0 Hz, J₂=8.8 Hz, 1H), 6.80 (d, J=8.8 Hz, 1H), 5.08 (d, J=5.6 Hz, 1H), 4.71-4.69 (m, 1H), 3.63 (t, J=4.8 Hz, 4H), 3.03 (t, J=5.2 Hz, 4H), 2.31-2.26 (m, 2H), 1.80-1.59 (m, 6H).

### Example 28

### 1-{5-[6,7-dichloro-4-(cyclopentylamino)phthalazin-1-yl]pyridin-2-yl}piperidin-4-ol

Example 28 was prepared using the procedure for the synthesis of Example 21, substituting piperidin-4-ol for 1-methylpiperazine. LCMS (Method C): m/z 458.1 (M+H), retention time: 1.923 minutes; ¹H NMR (400 MHz, CDCl₃) δ 8.45 (d, J=2.0 Hz, 1H), 8.18 (s, 1H), 7.90 (s, 1H), 7.87 (dd, J₁=2.4 Hz, J_{2=8.8} Hz, 1H), 6.84 (d, J=8.8 Hz, 1H), 5.01 (d, J=6.4 Hz, 1H), 4.71-4.69 (m, 1H), 4.20-4.16 (m, 2H), 3.99 (brs, 1H), 3.32-3.25 (m, 2H), 2.31-2.27 (m, 2H), 2.06-2.01 (m, 2H), 1.80-1.59 (m, 6H).

### Example 29

### 1-{4-[6,7-dichloro-4-(cyclopentylamino)phthalazin-1-yl]pyridin-2-yl}piperidin-4-ol

A mixture of Example 12 (50 mg, 0.133 mmol), piperidin-4-ol (134 mg, 13.25 mmol) and diisopropylamine (0.046 mL, 0.265 mmol) in dimethylsulfoxide (1 mL) was stirred at 100° C. for 16 hours. The mixture was purified by reverse phase preparative HPLC (Gilson 281: column Xbridge 21.2*250 mm c18 with a gradient 25-55% mobile phase B in mobile phase A; mobile phase A: water with 10 mM NH₄HCO₃; mobile phase B: acetonitrile) to give the title compound (26.2 mg, 0.057 mmol, 43.1% yield). LCMS (Method B): m/z 458.2 (M+H), retention time: 1.93 minutes; ¹H NMR (CDCl₃, 400 MHz) δ: 8.31(d, J=5.2 Hz, 1H), 8.08 (s, 1H), 7.90 (s,1H), 7.02 (s, 1H), 6.80 (d, J=5.2 Hz, 1H), 5.08 (d, J=6 Hz, 1H), 4.75-4.70 (m, 1H), 4.19-4.12 (m, 2H), 3.97-3.94 (m, 1H), 3.28-3.21 (m, 2H), 2.33-2.27 (m, 2H), 2.02-1.98 (m, 2H), 1.83-1.63 (m, 5H), 1.59-1.43 (m, 3H).

### Example 30

### 2-({5-[6,7-dichloro-4-(cyclopentylamino)phthalazin-1-yl]pyridin-2-yl}amino)ethanol

Example 30 was prepared using the procedure for the synthesis of Example 21, substituting 2-aminoethanol for 1-methylpiperazine. LCMS (Method C): m/z 418.1 (M+H), retention time: 1.815 minutes; ¹H NMR (400 MHz, DMSO-d₆) δ 8.83 (s, 1H), 8.20 (d, J=2.0 Hz, 1H), 7.92 (s, 1H), 7.64 (dd, J₁=2.4 Hz, J₂=8.8 Hz, 1H), 7.37 (d, J=6.4 Hz, 1H), 6.88-6.85 (t, J=5.6 Hz, 1H), 6.67-6.65 (d, J=8.4 Hz, 1H), 4.78-4.75 (m, 1H), 4.59-4.53 (m, 1H), 3.60-3.55 (m, 2H), 3.43-3.38 (m, 2H), 2.11-2.05 (m, 2H), 1.79-1.59 (m, 6H).

### Example 31

### 6,7-dichloro-N-cyclopentyl-4-[6-(pyrrolidin-1-yl)pyridin-3-yl]phthalazin-1-amine

Example 31 was prepared using the procedure for the synthesis of Example 21, substituting pyrrolodine for 1-methylpiperazine. LCMS (Method C): m/z 428.1 (M+H), retention time: 2.165 minutes; ¹H NMR (400 MHz, CDCl₃) δ 8.43 (d, J=2.4 Hz, 1H), 8.14 (s, 1H), 7.89 (s, 1H), 7.86 (dd, J₁=2.4 Hz, J₂=10.8 Hz, 1H), 6.53 (d, J=8.8 Hz, 1H), 4.99 (d, J=6.0 Hz, 1H), 4.72-4.69 (m, 1H), 3.55 (t, J=6.0 Hz, 4H), 2.31-2.27 (m, 2H), 2.08-2.04 (m, 4H), 1.79-1.56 (m, 6H).

### Example 32

### 6,7-dichloro-N-cyclopentyl-4-[2-(dimethylamino)pyridin-4-yl]phthalazin-1-amine

Example 32 was prepared using the procedure for the synthesis of Example 29, substituting dimethylamine for piperidin-4-ol. LCMS (Method B): m/z 402.1 (M+H), retention time: 2.12 minutes, ¹H NMR (CDCl₃, 400 MHz) δ: 8.30 (d, J=5.2 Hz, 1H), 8.10 (s, 1H), 7.91 (s, 1H), 6.85 (s, 1H), 6.75 (dd, J, =1.2 Hz, J₂=5.2 Hz, 1H), 5.09 (d, J=6.0 Hz, 1H), 4.76-4.70 (m, 1H), 3.15 (s, 6H), 2.32-2.26 (m, 2H), 1.83-1.56 (m, 6H).

### Example 33

### 4-[6-(3-aminopyrrolidin-1-yl)pyridin-3-yl]-6,7-dichloro-N-cyclopentylphthalazin-1-amine

Example 33 was prepared using the procedure for the synthesis of Example 21, substituting pyrrolidin-3-amine for 1-methylpiperazine. LCMS (Method C): m/z 443.2 (M+H), retention time: 1.847 minutes; ¹H NMR (400 MHz, CDCl₃) δ 8.43 (d, J=2.0 Hz, 1H), 8.12 (s, 1H), 7.90 (s, 1H), 7.85 (dd, J₁=2.4 Hz, J₂=10.8 Hz, 1H), 6.53 (d, J=8.4 Hz, 1H), 5.01 (d, J=6.0 Hz, 1H), 4.71-4.69 (m, 1H), 3.81-3.77 (m, 3H), 3.73-3.70 (m, 1H), 3.32-3.30 (m, 1H), 2.32-2.27 (m, 3H), 1.83-1.56 (m, 7H).

### Example 34

### 1-{5-[6,7-dichloro-4-(cyclopentylamino)phthalazin-1-yl]pyridin-2-yl}pyrrolidin-3-ol

Example 34 was prepared using the procedure for the synthesis of Example 21, substituting pyrrolidin-3-ol for 1-methylpiperazine. LCMS (Method C): m/z 444.2 (M+H), retention time: 1.876 minutes; ¹H NMR (400 MHz, CDCl₃) δ 8.43 (d, J=2.0 Hz, 1H), 8.12 (s, 1H), 7.92-7.84 (m, 2H), 6.54 (d, J=8.8 Hz, 1H), 5.02 (s, 1H), 4.71-4.67 (m, 2H), 3.74-3.64 (m, 4H), 2.31-2.27 (m, 2H), 2.21-2.17 (m, 2H), 1.789-1.55 (m, 6H).

### Example 35

### 2-[{4-[6,7-dichloro-4-(cyclopentylamino)phthalazin-1-yl]pyridin-2-yl}(methyl)amino]ethanol

Example 35 was prepared using the procedure for the synthesis of Example 29, substituting 2-(methylamino)ethanol for piperidin-4-ol. LCMS (Method B): m/z 432.1 (M+H), retention time: 2.11 minutes; ¹H NMR (CDCl₃, 400 MHz) δ: 8.20 (d, J=5.2 Hz, 1H), 8.08 (s, 1H), 7.90 (s, 1H), 6.89 (s, 1H), 6.80 (dd, J₁=1.2 Hz , J₂=5.2 Hz, 1H), 5.09 (d, J=6 Hz, 1H), 4.75-4.70 (m, 1H), 3.90 (t, J=4.8 Hz, 2H), 3.78 (t, J=4.8 Hz, 2H), 3.12 (s, 3H), 2.32-2.28 (m, 2H), 1.81-1.81 (m, 6H).

### Example 36

### 1-{4-[6,7-dichloro-4-(cyclopentylamino)phthalazin-1-yl]pyridin-2-yl}pyrrolidin-3-ol

Example 36 was prepared using the procedure for the synthesis of Example 29, substituting pyrrolidin-3-ol for piperidin-4-ol. LCMS (Method B): m/z 428.1 (M+H), retention time: 2.28 minutes; ¹H NMR (CDCl₃, 400 MHz) δ: 8.22 (d, J=5.2 Hz, 1H), 8.05 (s, 1H), 7.82 (s, 1H), 6.68-6.62 (m, 2H), 5.99 (d, J=5.2 Hz, 1H), 4.69-4.63 (m, 1H), 3.46-3.40 (t, J=6.4 Hz, 4H), 2.28-2.22 (m, 2H), 1.98-1.94 (m, 4H), 1.76-1.57 (m, 6H).

### Example 37

### methyl 6,7-dichloro-4-(cyclopentylamino)phthalazine-1-carboxylate

A mixture of PdCl₂(dppf) (0.231 g, 0.316 mmol), triethylamine (1.321 ml, 9.48 mmol) and Example 1 (1 g, 3.16 mmol) in N,N-dimethylformamide (25 mL) and methanol (25 mL) was stirred under carbon monoxide (4 atmosphere pressure) at 90° C. for 40 minutes. The reaction mixture was poured into ice-water (100 mL) and extracted with ethyl acetate (200 mL×2). The combined organic phase was washed with brine, dried over magnesium sulfate, filtered, and concentrated. The residue was purified by column chromatography on silica gel eluting with a gradient of petroleum ether/ethyl acetate from 4/1 to 2/1 to give title compound (800 mg, 2.116 mmol, 67.0% yield) as a white solid. LCMS (Method C): m/z 340.0 (M+H), retention time: 2.007 minutes; ¹H NMR (400 MHz, CDCl₃) δ 9.08 (s, 1H), 7.78 (s, 1H), 5.33 (d, J=6.4 Hz, 1H), 4.72-4.67 (m, 1H), 3.97 (s, 3H), 2.26-2.23 (m, 2H), 1.82-1.60 (m, 6H).

### Example 38

### 6,7-dichloro-4-(cyclopentylamino)phthalazine-1-carboxylic acid

To a solution of Example 37 (800 mg, 1.646 mmol) in tetrahydrofuran (3 mL), methanol (2 mL) and water (1 mL) was added lithium hydroxide hydrate (138 mg, 3.29 mmol). The mixture was stirred at room temperature for 3 hours. The pH of the mixture was adjusted to about 3 with the addition of 2N aqueous hydrochloric acid. The solid material was collected by filtration, and washed with water (10 mL) and petroleum ether/ethyl acetate (5/1, 10 mL) to give the title compound (740 mg, 2.269 mmol, 138% yield) as pale yellow solid. LCMS (Method C): m/z 362.1 (M+H), retention time: 1.487 minutes; ¹H NMR (400 MHz, DMSO-d₆) δ 8.84 (brs, 1H), 8.71 (d, J=6.0 Hz, 1H), 7.24 (d, J=2.8 Hz, 1H), 4.48-4.45 (m, 1H), 2.02-2.00 (m, 2H), 1.73-1.52 (m, 6H).

### Example 39

### 2-({4-[6,7-dichloro-4-(cyclopentylamino)phthalazin-1-yl]pyridin-2-yl}amino)ethanol

Example 39 was prepared using the procedure for the synthesis of Example 29, substituting 2-aminoethanol for piperidin-4-ol. LCMS (Method B): m/z 418.1 (M+H), retention time: 1.84 minutes. ¹H NMR (DMSO-d₆, 400 MHz) δ: 8.87 (s, 1H), 8.10 (d, J=5.2 Hz, 1H), 7.93 (s, 1H), 7.54 (d, J=5.2 Hz, 1H), 6.77-6.67 (m, 3H), 4.78-4.75 (m, 1H), 4.60-4.58 (m, 1H), 3.59-3.54 (m, 2H), 3.40-3.33 (m, 4H), 2.10-2.05 (m, 2H), 1.68-1.59 (m, 6H).

### Example 40

### 4-[2-(3-aminopyrrolidin-1-yl)pyridin-4-yl]-6,7-dichloro-N-cyclopentylphthalazin-1-amine

Example 40 was prepared using the procedure for the synthesis of Example 29, substituting pyrrolidin-3-amine for piperidin-4-ol. LCMS (Method B): m/z 443.2 (M+H), retention time: 1.84 minutes; ¹H NMR (DMSO-d₆, 400 MHz) δ: 8.29 (d, J=4.8 Hz, 1H), 8.10 (s, 1H), 7.90 (s, 1H), 6.75 (d, J=4.8 Hz, 1H), 6.69 (s, 1H), 5.07 (d, J=6 Hz, 1H), 4.76-4.70 (m, 1H), 3.77-3.57 (m, 4H), 3.29-3.26 (m, 1H), 2.32-2.22 (m, 4H), 1.87-1.50 (m, 6H).

### Example 41

### 6,7-dichloro-N-cyclopentyl-4-[6-(1,1-dioxidothiomorpholin-4-yl)pyridin-3-yl]phthalazin-1-amine

Example 41 was prepared using the procedure for the synthesis of Example 21, substituting thiomorpholine 1,1-dioxide for 1-methylpiperazine. LCMS (Method C): m/z 492.1 (M+H), retention time: 1.947 minutes; ¹H NMR (400 MHz, CDCl₃) δ 8.51 (d, J=2.0 Hz, 1H), 8.06 (s, 1H), 7.95 (d, J₁=1.6 Hz, J₂=8.8 Hz, 1H), 7.92 (s, 1H), 6.92 (d, J=8.8 Hz, 1H), 5.05 (d, J=6.4 Hz, 1H), 4.73-4.71 (m, 1H), 4.27 (d, J=5.2 Hz, 4H), 3.13 (t, J=5.2 Hz, 4H), 2.33-2.27 (m, 2H), 1.81-1.61 (m, 6H).

### Example 42

### 6,7-dichloro-4-(cyclopentylamino)phthalazine-1-carboxamide

To a solution of Example 38 (40 mg, 0.123 mmol) in N,N-dimethylformamide (2 mL) was added diisopropylethylamine (0.033 mL, 0.184 mmol) and HOBT (22.54 mg, 0.147 mmol) and then the mixture was stirred for 30 minutes. Ammonium chloride (52.5 mg, 0.981 mmol) and 1-ethyl-3-[3-(dimethylamino)propyl]-carbodiimide hydrochloride (28.2 mg, 0.147 mmol) were added. The reaction mixture was stirred at room temperature for 16 hours. The solution of the reaction was purified by reverse phase preparative HPLC (Gilson 281: column Xbridge 21.2*250 mm c18 with a gradient 25-55% mobile phase B in mobile phase A; mobile phase A: water with 10 mM NH₄HCO₃; mobile phase B:acetonitrile) to give the title compound (23.9 mg, 0.073 mmol, 59.9% yield) as a white solid. LCMS (Method C): m/z 325.1 (M+H), retention time: 1.851 minutes; ¹H NMR (400 MHz, DMSO-d₆) δ 9.37 (s, 1H), 8.87 (s, 1H), 8.30 (s, 1H), 7.97 (brs, 1H), 7.61 (s, 1H), 4.63-4.58 (m, 1H), 2.10-2.05 (m, 2H), 1.77-1.60 (m, 6H).

### Example 43

### tert-butyl {4-[6,7-dichloro-4-(cyclopentylamino)phthalazin-1-yl]-1H-pyrazol-1-yl}acetate

### Example 43a

### tert-butyl 2-(4-(4,4,5,5-tetramethyl-1,3,2-dioxaborolan-2-yl)-1H-pyrazol-1-yl)acetate

A mixture of 4-(4,4,5,5-tetramethyl-1,3,2-dioxaborolan-2-yl)-1H-pyrazole (2.0 g, 10.31 mmol), tert-butyl 2-bromoacetate (2.212 g, 11.34 mmol) and K₂CO₃ (1.709 g, 12.37 mmol) in acetone (20 mL) was stirred at 65° C. for 13 hours under nitrogen. The reaction was poured into ice water (20 mL) and extracted with ethyl acetate (50 mL×2). The combined organic phase was washed with brine, dried over magnesium sulfate, filtered and concentrated under reduced pressure. The residue was purified by column chromatography on silica gel (petroleum ether/ethyl acetate=5/1) to give the title compound (3 g, 8.76 mmol, 85% yield) as an oil. LCMS (Method C): m/z 309.2 (M+H), retention time: 1.946 minutes; ¹H NMR (400 MHz, CDCl₃) δ 7.82 (s, 1H), 7.75 (s, 1H), 4.82 (s, 1H), 1.47 (s, 9H), 1.28 (s, 12H).

### Example 43b

### tert-butyl {4-[6,7-dichloro-4-(cyclopentylamino)phthalazin-1-yl]-1H-pyrazol-1-yl}acetate

A mixture of Example 1c (2 g, 6.32 mmol), Example 43a (2.336 g, 7.58 mmol), tetrakis(triphenylphosphine)palladium(0) (0.730 g, 0.632 mmol) and K₂CO₃ (2.62 g, 18.95 mmol) in 1,4-dioxane/water (ratio: 4:1, 8 mL) was stirred at 80° C. for 3 hours under nitrogen. The reaction mixture was poured into ice water (25 mL) and extracted with ethyl acetate (60 mL×3). The combined organic phase was washed with brine, dried over magnesium sulfate, filtered, and concentrated under reduced pressure. The residue was purified by column chromatography on silica gel to give the title compound (2 g, 3.68 mmol, 58.2% yield). LCMS (Method C): m/z 462.2 (M+H), retention time: 2.067 minutes; ¹H NMR (400 MHz, DMSO-d₆) δ 8.85 (s, 1H), 8.29 (s, 1H), 8.15 (s, 1H), 7.93 (s, 1H), 7.38 (d, J=6.4 Hz, 1H), 5.07 (s, 2H), 4.57-4.54 (m, 1H), 2.09-2.04 (m, 2H), 1.77-1.58 (m, 6H), 1.46 (s, 9H).

### Example 44

### {4-[6,7-dichloro-4-(cyclopentylamino)phthalazin-1-yl]-1H-pyrazol-1-yl}acetic acid

A solution of Example 43b (2 g, 4.33 mmol) in dichloromethane (30 mL) and trifluoroacetic acid (15 mL) was stirred at room temperature for 3 hours. The reaction mixture was concentrated under reduced pressure. The solid residue was triturated in hot ethyl acetate (50 mL) to give the title compound (1.2 g, 2.95 mmol, 68.3% yield). LCMS (Method C): m/z 406.1 (M+H), retention time: 1.546 minutes; ¹H NMR (400 MHz, DMSO-d₆) δ 9.04 (s, 1H), 8.41 (s, 1H), 8.26 (s, 1H), 7.99 (s, 1H), 5.11 (s, 2H), 4.46-4.44 (m, 1H), 2.13-2.08 (m, 2H), 1.80-1.60 (m, 6H).

### Example 45

### 6,7-dichloro-4-(cyclopentylamino)-N-ethylphthalazine-1-carboxamide

To a solution of Example 38 (40 mg, 0.123 mmol) in N,N-dimethylformamide (2 mL) was added diisopropylethylamine (0.054 ml, 0.307 mmol) and HATU (56.0 mg, 0.147 mmol) and ethanamine hydrochloride (15.00 mg, 0.184 mmol). The mixture was stirred at room temperature for 16 hours. The reaction solution was purified by reverse phase preparative HPLC (Gilson 281: column Xbridge 21.2*250 mm c18 with a gradient 25-55% mobile phase B in mobile phase A; mobile phase A: water with 10 mM NH₄HCO₃; mobile phase B:acetonitrile) to give the title compound (23.6 mg, 0.067 mmol, 54.5% yield) as a solid. LCMS (Method C): m/z 353.1 (M+H), retention time: 2.060 minutes; ¹H NMR (400 MHz, CDCl₃) δ 9.77 (s, 1H), 8.12 (d, J=5.6 Hz, 1H), 7.82 (s, 1H), 5.31 (d, J=6.4 Hz, 1H), 4.72-4.67 (m, 1H), 3.55-3.48 (q, J=7.2 Hz, 2H), 2.31-2.25 (m, 2H), 1.84-1.60 (m, 6H), 1.26 (t, J=7.2 Hz, 3H).

### Example 46

### 6,7-dichloro-4-(cyclopentylamino)-N-(2-hydroxyethyl)-N-methylphthalazine-1-carboxamide

Example 46 was prepared using the procedure for the synthesis of Example 42, substituting 2-(methylamino)ethanol for ammonium chloride. LCMS (Method C): m/z 383.2 (M+H), retention time: 1.723 minutes; ¹H NMR (400 MHz, CDCl₃) δ 8.24 (s, 1H), 7.79 (s, 1H), 5.35 (s, 1H), 5.21-5.20 (m, 1H), 4.64-4.62 (m, 1H), 3.92-3.82 (m, 2H), 3.57-3.53 (m, 2H), 3.20 (s, 3H), 2.19-2.15 (m, 2H), 1.77-1.60 (m, 6H).

### Example 47

### [6,7-dichloro-4-(cyclopentylamino)phthalazin-1-yl](pyrrolidin-1-yl)methanone

Example 47 was prepared using the procedure for the synthesis of Example 45, substituting pyrrolidine for ethylamine hydrochloride. LCMS (Method C): m/z 379.1 (M+H), retention time: 1.928 minutes; ¹H NMR (400 MHz, CDCl₃) δ 8.39 (s, 1H), 7.77 (s, 1H), 5.11 (d, J=6.4 Hz, 1H), 4.68-4.62 (m, 1H), 3.70 (t, J=6.8 Hz, 2H), 3.64 (t, J=6.8 Hz, 2H), 2.23-2.16 (m, 2H), 1.96-1.82 (m, 5H), 1.73-1.60 (m, 5H).

### Example 48

### [6,7-dichloro-4-(cyclopentylamino)phthalazin-1-yl](3-hydroxypyrrolidin-1-yl)methanone

Example 48 was prepared using the procedure for the synthesis of Example 42, substituting pyrrolidin-3-ol for ammonium chloride. LCMS (Method C): m/z 395.1 (M+H), retention time: 1.709 minutes; ¹H NMR (400 MHz, CDCl₃) δ 8.46 (d, J=25.2 Hz, 1H), 7.84 (d, J=3.6 Hz, 1H), 5.25-5.24 (m, 1H), 4.72-4.70 (m, 1H), 4.61-4.51 (m, 1H), 3.99-3.85 (m, 4H), 3.68 (d, J=12.4 Hz, 1H) 2.35-2.32 (m, 2H), 2.15-2.00 (m, 2H), 1.81-1.65 (m, 6H).

### Example 49

### [6,7-dichloro-4-(cyclopentylamino)phthalazin-1-yl](4-methylpiperazin-1-yl)methanone

Example 49 was prepared using the procedure for the synthesis of Example 45, substituting 1-methylpiperazine for ethylamine hydrochloride. LCMS (Method C): m/z 408.2 (M+H), retention time: 1.797 minutes; ¹H NMR (400 MHz, CDCl₃) δ 8.14 (s, 1H), 7.86 (s, 1H), 5.28 (d, J=6.4 Hz, 1H), 4.72-4.66 (m, 1H), 3.93 (t, J=5.2 Hz, 2H), 3.60 (t, J=5.2 Hz, 2H), 2.57 (t, J=4.8 Hz, 2H), 2.44 (t, J=4.8 Hz, 2H), 2.33 (s, 3H), 2.27-2.21 (m, 2H), 1.82-1.50 (m, 6H).

### Example 50

### [6,7-dichloro-4-(cyclopentylamino)phthalazin-1-yl](1,1-dioxidothiomorpholin-4-yl)methanone

Example 50 was prepared using the procedure for the synthesis of Example 45, substituting thiomorpholine 1,1-dioxide for ethylamine hydrochloride. ¹H NMR (400 MHz, CDCl₃) δ 8.15 (s, 1H), 7.81 (s, 1H), 5.23 (d, J=6.8 Hz, 1H), 4.68-4.63 (m, 1H), 4.31 (s, 2H), 3.99 (t, J=5.2 Hz, 2H), 3.31 (s, 2H), 3.16 (t, J=5.2 Hz, 2H), 2.23-2.17 (m, 2H), 1.76-1.63 (m, 6H).

### Example 51

### [6,7-dichloro-4-(cyclopentylamino)phthalazin-1-yl](morpholin-4-yl)methanone

Example 51 was prepared using the procedure for the synthesis of Example 45, substituting morpholine for ethylamine hydrochloride. LCMS (Method C): m/z 395.1 (M+H), retention time: 1.836 minutes; ¹H NMR (400 MHz, CDCl₃) δ 8.19 (s, 1H), 7.92 (s, 1H), 5.52 (brs, 1H), 4.72-4.67 (m, 1H), 3.93-3.83 (m, 4H), 3.74-3.63 (m, 4H), 2.27-2.21 (m, 2H), 1.79-1.60 (m, 6H).

### Example 52

### [6,7-dichloro-4-(cyclopentylamino)phthalazin-1-yl](4-hydroxypiperidin-1-yl)methanone

Example 52 was prepared using the procedure for the synthesis of Example 42, substituting piperidin-4-ol for ammonium chloride. LCMS (Method C): m/z 409.1 (M+H), retention time: 1.720 minutes; ¹H NMR (400 MHz, CDCl₃) δ 8.13 (s, 1H), 7.86 (s, 1H), 5.23-5.21 (m, 1H), 4.73-4.68 (m, 1H), 4.30-4.26 (t, J=6.4 Hz, 1H), 4.02 (t, J=6.6 Hz, 1H), 3.81-3.78 (m, 1H), 3.59 (t, J=8.0 Hz, 1H), 3.37-3.31 (m, 1H), 2.23-1.70 (m, 13H).

### Example 53

### 6,7-dichloro-4-(cyclopentylamino)-N-phenylphthalazine-1-carboxamide

Example 53 was prepared using the procedure for the synthesis of Example 45, substituting aniline for ethylamine hydrochloride. LCMS (Method C): m/z 401.1 (M+H), retention time: 2.281 minutes; ¹H NMR (400 MHz, CDCl₃) δ 10.16 (s, 1H), 9.84 (s, 1H), 7.85 (s, 1H), 7.75 (d, J=7.6 Hz, 2H), 7.39 (d, J=8.0 Hz, 2H), 7.15 (t, J=7.2 Hz, 1H), 5.41 (d, J=6.8 Hz, 1H), 4.77-4.72 (m, 1H), 2.35-2.28 (m, 2H), 1.76-1.63 (m, 6H).

### Example 54

### 6,7-dichloro-4-(cyclopentylamino)-N-(1-methyl-1H-pyrazol-4-yl)phthalazine-1-carboxamide

Example 54 was prepared using the procedure for the synthesis of Example 45, substituting 1-methyl-1H-pyrazol-4-amine for ethylamine hydrochloride. LCMS (Method C): m/z 405.1 (M+H), retention time: 1.956 minutes; ¹H NMR (400 MHz, CDCl₃) δ 10.00 (s, 1H), 9.81 (s, 1H), 8.04 (s, 1H), 7.86 (s, 1H), 7.55 (s, 1H), 5.44 (d, J=5.2 Hz, 1H), 4.75-4.70 (m, 1H), 3.93 (s, 3H), 2.33-2.28 (m, 2H), 1.86-1.72 (m, 6H).

### Example 55

### 2-{4-[6,7-dichloro-4-(cyclopentylamino)phthalazin-1-yl]-1H-pyrazol-1-yl}-N-ethyl acetamide

To a solution of Example 44 (50 mg, 0.123 mmol) in N,N-dimethylformamide (2 mL) was added diisopropylethylamine (0.129 mL, 0.738 mmol), HATU (70.2 mg, 0.185 mmol) and ethylamine hydrochloride (30.1 mg, 0.369 mmol). The mixture was stirred at room temperature for 13 hours. The reaction solution was purified by reverse phase preparative HPLC (Gilson 281: column Xbridge 21.2*250 mm c18 with a gradient 25-55% mobile phase B in mobile phase A; mobile phase A: water with 10 mM NH₄HCO₃; mobile phase B:acetonitrile) to give the title compound (13.5 mg, 0.031 mmol, 25.3% yield) as a solid. LCMS (Method C): m/z 433.1 (M+H), retention time: 1.792 minutes; ¹H NMR (400 MHz, DMSO-d₆) δ 8.84 (s, 1H), 8.26 (s, 1H), 8.19 (s, 1H), 8.17 (s, 1H), 7.91 (s, 1H), 7.37 (d, J=6.0 Hz, 1H), 4.88 (s, 2H), 4.57-4.54 (m, 1H), 3.15 (q, J=7.2 Hz, 2H), 2.11-2.06 (m, 2H), 1.77-1.62 (m, 6H), 1.06 (t, J=7.2 Hz, 2H).

### Example 56

### 2-{4-[6,7-dichloro-4-(cyclopentylamino)phthalazin-1-yl]-1H-pyrazol-1-yl}-N-(2-hydroxyethyl)acetamide

Example 56 was prepared using the procedure for the synthesis of Example 55, substituting 2-aminoethanol for ethylamine hydrochloride. LCMS (Method C): m/z 449.2 (M+H), retention time: 1.673 minutes; ¹H NMR (400 MHz, DMSO-d₆) δ 8.84 (s, 1H), 8.26 (s, 1H), 8.21 (s, 1H), 8.19 (s, 1H), 7.91 (s, 1H), 7.37 (d, J=6.4 Hz, 1H), 4.92 (s, 2H), 4.75 (t, J=5.2 Hz, 2H), 4.57-4.54 (m, 1H), 3.47-3.42 (dd, J₁=6.0 Hz, J₂=11.6 Hz, 2H), 3.22-3.18 (q, J=6.0 Hz, 2H), 2.11-2.06 (m, 2H), 1.77-1.59 (m, 6H).

### Example 57

### 2-{4-[6,7-dichloro-4-(cyclopentylamino)phthalazin-1-yl]-1H-pyrazol-1-yl}-N-(2-hydroxyethyl)-N-methylacetamide

Example 57 was prepared using the procedure for the synthesis of Example 55, substituting 2-(methylamino)ethanol for ethylamine hydrochloride. LCMS (Method C): m/z 463.2 (M+H), retention time: 1.705 minutes; ¹H NMR (400 MHz, CD₃OD) 6 8.65 (s, 1H), 8.34 (d, J=1.2 Hz, 1H), 8.12 (d, J=4.8 Hz, 1H), 7.95 (d, J=4.8 Hz, 1H), 5.40 (s, 1H), 5.3 (s, 1H), 4.60-4.58 (m, 1H), 3.83-3.73 (m, 2H), 3.65-3.56 (m, 2H), 3.253-3.05 (d, J=79.2 Hz, 3H). 2.22-2.18 (m, 2H), 1.85-1.71 (m, 6H).

### Example 58

### 2-{4-[6,7-dichloro-4-(cyclopentylamino)phthalazin-1-yl]-1H-pyrazol-1-yl}-1-(pyrrolidin-1-yl)ethanone

Example 58 was prepared using the procedure for the synthesis of Example 55, substituting pyrrolidine for ethylamine hydrochloride. LCMS (Method C): m/z 459.2 (M+H), retention time: 1.835 minutes; ¹H NMR (400 MHz, DMSO-d₆) δ 8.85 (s, 1H), 8.21 (s, 1H), 8.18 (s, 1H), 7.89 (s, 1H), 7.38 (d, J=6.8 Hz, 1H), 5.15 (s, 2H), 4.57-4.55 (m, 1H), 3.56-3.42 (m, 4H), 2.10-2.05 (m, 2H), 1.96-1.92 (m, 2H), 1.69-1.64 (m, 4H), 1.63-1.60 (m, 4H).

### Example 59

### 2-{4-[6,7-dichloro-4-(cyclopentylamino)phthalazin-1-yl]-1H-pyrazol-1-yl}-1-(3-hydroxypyrrolidin-1-yl)ethanone

Example 59 was prepared using the procedure for the synthesis of Example 55, substituting pyrrolidin-3-ol for ethylamine hydrochloride. LCMS (Method C): m/z 475.1 (M+H), retention time: 1.692 minutes; ¹H NMR (400 MHz, DMSO-d₆) δ 8.85 (s, 1H), 8.22 (s, 1H), 8.17 (s, 1H), 7.89 (s, 1H), 7.37 (d, J=6.4 Hz, 1H), 5.17-5.00 (m, 3H), 4.56-4.53 (m, 1H), 4.38-4.28 (d, J=39.6 Hz, 1H), 3.65-3.60 (m, 4H), 2.10-2.05 (m, 2H), 1.90-1.58 (m, 8H).

### Example 60

### 2-{4-[6,7-dichloro-4-(cyclopentylamino)phthalazin-1-yl]-1H-pyrazol-1-yl}-1-(1,3-oxazolidin-3-yl)ethanone

Example 60 was prepared using the procedure for the synthesis of Example 55, substituting oxazoline for ethylamine hydrochloride. LCMS (Method C): m/z 462 (M+H), retention time: 1.752 minutes; ¹H NMR (400 MHz, DMSO-d₆) δ 8.84 (s, 1H), 8.55 (d, J=6.8 Hz, 1H), 8.25 (s, 1H), 8.18 (s, 1H), 7.90 (s, 1H), 7.37 (d, J=6.8 Hz, 1H), 4.90 (s, 2H), 4.57-4.55 (m, 1H), 4.28 (d, J=2.8 Hz, 1H), 3.84-3.69 (m, 4H), 3.53-3.50 (dd, J₁=3.6 Hz, J₂=9.2 Hz, 1H), 2.16-2.05 (m, 3H), 1.79-1.59 (m, 7H).

### Example 61

### 2-{4-[6,7-dichloro-4-(cyclopentylamino)phthalazin-1-yl]-1H-pyrazol-1-yl}-1-(4-methylpiperazin-1-yl)ethanone

Example 61 was prepared using the procedure for the synthesis of Example 55, substituting 1-methylpiperazine for ethylamine hydrochloride. LCMS (Method C): m/z 488.2 (M+H), retention time: 1.745 minutes; ¹H NMR (400 MHz, CDCl₃) δ 8.29 (s, 1H), 8.03 (s, 1H), 7.99 (s, 1H), 7.88 (s, 1H), 5.11 (s, 2H), 4.97 (d, J=6.4 Hz, 1H), 4.70-4.67 (m, 1H), 3.67 (t, J=4.8 Hz, 2H), 3.61 (t, J=4.8 Hz, 2H), 2.42 (s, 4H), 2.32 (s, 3H), 2.29-2.26 (m, 3H), 1.80-1.60 (m, 4H).

### Example 62

### 1-(4-acetylpiperazin-1-yl)-2-{4-[6,7-dichloro-4-(cyclopentylamino)phthalazin-1-yl]-1H-pyrazol-1-yl}ethanone

Example 62 was prepared using the procedure for the synthesis of Example 55, substituting 1-(piperazin-1-yl)ethanone for ethylamine hydrochloride. LCMS (Method C): m/z 516.1 (M+H), retention time: 1.702 minutes; ¹H NMR (400 MHz, CDCl₃) δ 8.28 (s, 1H), 8.03 (s, 1H), 7.99 (s, 1H), 7.89 (s, 1H), 5.13 (s, 2H), 4.98 (brs, 1H), 4.70-4.67 (m, 1H), 3.71-3.62 (m, 6H), 3.51-3.50 (m, 2H), 2.30-2.22 (m, 2H), 2.13 (s, 3H), 2.02-1.99 (m, 1H), 1.81-1.71 (m, 4H).

### Example 63

### 2-{4-[6,7-dichloro-4-(cyclopentylamino)phthalazin-1-yl]-1H-pyrazol-1-yl}-1-(1,1-dioxidothiomorpholin-4-yl)ethanone

Example 63 was prepared using the procedure for the synthesis of Example 55, substituting thiomorpholine 1,1-dioxide for ethylamine hydrochloride. LCMS (Method C): m/z 523.2 (M+H), retention time: 1.751 minutes; ¹H NMR (400 MHz, DMSO-d₆) δ 8.85 (s, 1H), 8.17 (d, J=6.4 Hz, 2H), 7.91 (s, 1H), 7.38 (d, J=6.4 Hz, 1H), 5.38 (s, 2H), 4.57-4.55 (m, 1H), 3.96-3.92 (t, J=8.0 Hz, 4H), 3.36-3.31 (m, 2H), 3.16 (s, 2H), 2.09-2.05 (m, 2H), 1.77-1.588 (m, 6H).

### Example 64

### 2-{4-[6,7-dichloro-4-(cyclopentylamino)phthalazin-1-yl]-1H-pyrazol-1-yl}-1-(4-hydroxypiperidin-1-yl)ethanone

Example 64 was prepared using the procedure for the synthesis of Example 55, substituting piperidin-4-ol for ethylamine hydrochloride. LCMS (Method C): m/z 489.2 (M+H), retention time: 1.701 minutes; ¹H NMR (400 MHz, DMSO-d₆) δ 8.84 (s, 1H), 8.21 (s, 1H), 8.17 (s, 1H), 7.88 (s, 1H), 7.37 (d, J=6.4 Hz, 1H), 5.25 (d, J=2.8 Hz, 2H), 4.815 (d, J=4.0 Hz, 1H), 4.60-4.50 (m, 1H), 3.91-3.89 (m, 1H), 3.72-3.71 (m, 2H), 3.32-3.326 (m, 2H), 2.09-2.05 (m, 2H), 1.76-1.61 (m, 8H), 1.38-1.1.36 (m, 2H).

### Example 65

### 2-{4-[6,7-dichloro-4-(cyclopentylamino)phthalazin-1-yl]-1H-pyrazol-1-yl}-1-(morpholin-4-yl)ethanone

Example 65 was prepared using the procedure for the synthesis of Example 55, substituting morpholine for ethylamine hydrochloride. LCMS (Method C): m/z 475.2 (M+H), retention time: 1.769 minutes; ¹H NMR (400 MHz, DMSO-d₆) δ 8.85 (s, 1H), 8.21 (s, 1H), 8.17 (s, 1H), 7.90 (s, 1H), 7.37 (d, J=6.8 Hz, 1H), 5.27 (s, 2H), 4.62-4.58 (m, 1H), 3.66-3.42 (m, 8H), 2.07-2.01 (m, 2H), 1.77-1.60 (m, 6H).

### Example 66

### 6,7-dichloro-4-(cyclopentylamino)-N-(2-hydroxyethyl)phthalazine-1-carboxamide

Example 66 was prepared using the procedure for the synthesis of Example 42, substituting 2-aminoethanol for ammonium chloride LCMS (Method C): m/z 369.1 (M+H), retention time: 1.791 minutes; ¹H NMR (400 MHz, CDCl₃) δ 9.64 (s, 1H), 8.42 (s, 1H), 7.76 (s, 1H), 5.28 (t, J=4.8 Hz, 2H), 4.65-4.62 (m, 1H), 3.80 (s, 2H), 3.61-3.57 (m, 2H), 2.20-2.10 (m, 2H), 1.77-1.60 (m, 6H).

### Example 67

### 6,7-dichloro-N-cyclopentyl-4-[1-(methylsulfonyl)-1,2,3,6-tetrahydropyridin-4-yl]phthalazin-1-amine

To a solution of Example 24 (50 mg, 0.138 mmol) in N,N-dimethylformamide (2 mL) was added diisopropylethylamine (0.060 mL, 0.344 mmol) and methanesulfonyl chloride (18.92 mg, 0.165 mmol) at 0° C. The mixture was stirred at room temperature for 5 hours. Water (0.5 mL) was added to the reaction mixture, which was directly purified by reverse phase preparative HPLC (Gilson 281: column Xbridge 21.2*250 mm c18 with a gradient 25-55% mobile phase B in mobile phase A; mobile phase A: water with 10 mM NH₄HCO₃; mobile phase B:acetonitrile) to give the title compound (12.7 mg, 0.029 mmol, 20.91% yield). LCMS (Method B): m/z 441.3 (M+H), retention time: 1.91 minutes; ¹H NMR (CD₃OD, 400 MHz) δ 8.51 (s, 1H), 8.07 (s, 1H), 5.95 (d, J=1.6 Hz, 1H), 4.45-4.40 (m, 1H), 3.97-3.95 (m, 2H), 3.49 (t, J=6.0 Hz, 2H), 2.87 (s, 3H), 2.65-2.55 (m, 2H), 2.08-2.05 (m, 2H), 1.72-1.58 (m, 6H).

### Example 68

### 6,7-dichloro-N-cyclopentyl-4-[1-(cyclopropylsulfonyl)-1,2,3,6-tetrahydropyridin-4-yl]phthalazin-1-amine

Example 68 was prepared using the procedure for the synthesis of Example 67, substituting cyclopropanesulfonyl chloride for mathanesulfonyl chloride. LCMS (Method B): m/z 467 (M+H), retention time: 1.985 minutes; ¹H NMR (CD₃OD, 400 MHz) δ 8.63 (s, 1H), 8.18 (s, 1H), 6.05 (s, 1H), 4.64-4.56 (m, 2H), 4.16-4.13 (m, 2H), 3.69-3.66 (m, 2H), 2.75-2.64 (m, 3H), 2.17-2.05 (m, 2H), 1.84-1.69 (m, 6H), 1.12-1.09 (m, 4H).

### Example 69

### 6,7-dichloro-N-cyclopentyl-4-[1-(phenylsulfonyl)-1,2,3,6-tetrahydropyridin-4-yl]phthalazin-1-amine

Example 69 was prepared using the procedure for the synthesis of Example 67, substituting benzenesulfonyl chloride for mathanesulfonyl chloride. LCMS (Method B): m/z 503.2 (M+H), retention time: 2.095 minutes; ¹H NMR (CD₃OD, 400 MHz) δ 8.61 (s, 1H), 8.02 (s, 1H), 7.94-7.92 (m, 2H), 7.73-7.66 (m, 3H), 5.95(s, 1H), 4.55-4.52 (m, 1H), 3.93 (t, J=2.4 Hz, 2H), 3.48 (t, J=1.6 Hz, 2H), 2.69-2.67 (m, 2H), 2.18-2.14 (m, 2H), 1.71-1.64 (m, 6H).

### Example 70

### cyclopropyl {4-[6,7-dichloro-4-(cyclopentylamino)phthalazin-1-yl]-3,6-dihydropyridin-1(2H)-yl}methanone

Example 70 was prepared using the procedure for the synthesis of Example 67, substituting cyclopropanecarbonyl chloride for mathanesulfonyl chloride. LCMS (Method B): m/z 431.2 (M+H), retention time: 1.93 minutes; ¹H NMR (CD₃OD, 400 MHz) δ 8.62 (s, 1H), 8.17 (s, 1H), 6.05 (d, J=6.4 Hz, 1H), 4.57 (d, J=4.8 Hz, 2H), 4.34 (s, 1H), 4.10 (t, J=5.2 Hz, 1H), 3.93 (d, J=4.4 Hz, 1H), 2.78 (s, 1H), 2.65 (s, 1H), 2.18-2.15 (m, 3H), 1.84-1.69 (m, 6H), 0.96 (d, J=6.0 Hz, 4H).

### Example 71

### 1-{4-[6,7-dichloro-4-(cyclopentylamino)phthalazin-1-yl]-3,6-dihydropyridin-1(2H)-yl}-2-hydroxypropan-1-one

Example 71 was prepared using the procedure for the synthesis of Example 72, substituting 2-hydroxypropanoic acid for tetrahydrofuran-3-carboxylic acid. LCMS (Method B): m/z 435.2 (M+H), retention time: 1.82 minutes; ¹H NMR (CD₃OD, 400 MHz) δ 8.51 (s, 1H), 8.06 (s, 1H), 5.92 (d, J=14.0 Hz, 1H), 4.60-4.57 (m, 1H), 4.47-4.43 (m, 1H), 4.26-4.20 (m, 2H), 3.81-3.78 (m, 2H), 2.63-2.55 (m, 2H), 2.08-2.03 (m, 2H), 1.74-1.58 (m, 6H), 1.35-1.29 (m, 3H).

### Example 72

### {4-[6,7-dichloro-4-(cyclopentylamino)phthalazin-1-yl]-3,6-dihydropyridin-1(2H)-yl}(tetrahydrofuran-3-yl)methanone

To a solution of Example 24 (50 mg, 0.138 mmol) in N, N-dimethylacetamide (2 mL) was added diisopropylethylamine (0.060 ml, 0.344 mmol), HATU (62.8 mg, 0.165 mmol) and tetrahydrofuran-3-carboxylic acid (15.98 mg, 0.138 mmol). The mixture was stirred at room temperature overnight. The reaction solution was purified by reverse phase preparative HPLC (Gilson 281: column Xbridge 21.2*250 mm c18 with a gradient 25-55% mobile phase B in mobile phase A; mobile phase A: water with 10 mM NH₄HCO₃; mobile phase B: acetonitrile) to give the title compound (11 mg, 0.024 mmol, 17.15% yield). LCMS (Method B): m/z 461.2 (M+H), retention time: 1.85 minutes; ¹H NMR (CD₃OD, 400 MHz) δ 8.51 (s, 1H), 8.07-8.06 (d, J=4.4 Hz, 1H), 5.93 (d, J=8.0 Hz, 1H), 4.47-4.23 (m, 3H), 3.91-3.70 (m, 6H), 3.47-3.45 (m, 1H), 2.63-2.53 (m, 2H), 2.14-2.04 (m, 4H), 1.72-1.58 (m, 6H).

### Example 73

### 3-{4-[6,7-dichloro-4-(cyclopentylamino)phthalazin-1-yl]-3,6-dihydropyridin-1(2H)-yl}-3-oxopropanenitrile

Example 73 was prepared using the procedure for the synthesis of Example 72, substituting 2-cyanoacetic acid for tetrahydrofuran-3-carboxylic acid. LCMS (Method B): m/z 430.2 (M+H), retention time: 1.84 minutes; ¹H NMR (CD₃OD, 400 MHz) δ 8.52 (s, 1H), 8.09 (d, J=8.8 Hz, 1H), 5.91 (d, J=20.4 Hz, 1H), 4.47-4.43 (m, 1H), 4.24 (d, J=2.8 Hz, 1H), 4.16 (d, J=2.8 Hz, 1H), 3.82 (t, J=6.0 Hz, 1H), 3.67 (t, J=5.6 Hz, 1H), 2.65-2.55 (m, 2H), 2.09-2.02 (m, 2H), 1.72-1.58 (m, 6H).

### Example 74

### {4-[6,7-dichloro-4-(cyclopentylamino)phthalazin-1-yl]-3,6-dihydropyridin-1(2H)-yl}(phenyl)methanone

Example 74 was prepared using the procedure for the synthesis of Example 72, substituting benzoic acid for tetrahydrofuran-3-carboxylic acid. LCMS (Method B): m/z 467.2 (M+H), retention time: 2.02 minutes; ¹H NMR (CD₃OD, 400 MHz) δ 8.63 (s, 1H), 8.24 and 8.14 (s, 1H), 7.53 (s, 5H), 6.12 and 5.90 (s, 1H), 4.60-4.50 (m, 5H), 3.77 and 3.33 (s, 1H), 2.71 (s, 2H), 2.17 (s, 2H), 1.84-1.69 (m, 6H).

### Example 75

### {4-[6,7-dichloro-4-(cyclopentylamino)phthalazin-1-yl]-3,6-dihydropyridin-1(2H)-yl}(2-methylphenyl)methanone

Example 75 was prepared using the procedure for the synthesis of Example 72, substituting 2-methylbenzoic acid for tetrahydrofuran-3-carboxylic acid. LCMS (Method B): m/z 481.2 (M+H), retention time: 2.05 minutes; ¹H NMR (CD₃OD, 400 MHz) δ 8.63 (s, 1H), 8.22 and 8.12 (s, 1H), 7.41-7.30 (m, 4H), 6.13 and 5.90 (s, 1H), 4.60 (m, 8H), 3.62 (t, J=6.0 Hz, 1H), 2.78-2.60 (m, 1H), 2.39-2.37 (m, 1H), 2.38 (d, J=6.4 Hz, 3H), 2.20-2.15 (m, 2H), 1.84-1.69 (m, 6H).

### Example 76

### {4-[6,7-dichloro-4-(cyclopentylamino)phthalazin-1-yl]-3,6-dihydropyridin-1(2H)-yl}(2,6-dimethylphenyl)methanone

Example 76 was prepared using the procedure for the synthesis of Example 72, substituting 2,6-dimethylbenzoic acid for tetrahydrofuran-3-carboxylic acid. LCMS (Method B): m/z 495.3 (M+H), retention time: 2.10 minutes; ¹H NMR (CD₃OD, 400 MHz) δ 8.52 (s, 1H), 8.08-7.99 (d, J=38.0 Hz, 1H), 7.16-7.02 (m, 3H), 6.05-5.80 (d, J=96.8 Hz, 1H), 4.46 (d, J=2.8 Hz, 2H), 4.04 (t, J=6.4 Hz, 1H), 3.88 (d, J=2.8 Hz, 1H), 3.46 (t, J=6.0 Hz, 1H), 2.68 (d, J=2.0 Hz, 1H), 2.50 (d, J=5.6 Hz, 1H), 2.20 (t, J=10.0 Hz, 6H), 2.07 (d, J=6.8 Hz, 2H), 1.72-1.58 (m, 7H).

### Example 77

### {4-[6,7-dichloro-4-(cyclopentylamino)phthalazin-1-yl]-3,6-dihydropyridin-1(2H)-yl}(pyridin-3-yl)methanone

Example 77 was prepared using the procedure for the synthesis of Example 72, substituting nicotinic acid for tetrahydrofuran-3-carboxylic acid. LCMS (Method B): m/z 468.2 (M+H), retention time: 1.83 minutes; ¹H NMR (CD₃OD, 400 MHz) δ 8.64 (s, 1H), 8.58-8.57 (dd, J₁=1.6 Hz, J₂=9.2 Hz, 1H), 8.52 (s, 1H), 8.14-8.06 (m, 1H), 7.92 (d, J=7.2 Hz, 1H), 7.49-7.46 (dd, J₁=5.2 Hz, J₂=7.6 Hz, 1H) , 6.01-5.80 (d, J=83.2 Hz ,1H), 4.48-4.41 (m, 2H), 4.17-4.00 (m, 2H), 3.70-3.63 (m, 1H), 2.64-2.55 (m, 2H), 2.11-2.03 (m, 2H), 1.73-1.58 (m, 7H).

### Example 78

### {4-[6,7-dichloro-4-(cyclopentylamino)phthalazin-1-yl]-3,6-dihydropyridin-1(2H)-yl}(1-methyl-1H-pyrazol-4-yl)methanone

Example 78 was prepared using the procedure for the synthesis of Example 72, substituting 1-methyl-1H-pyrazole-4-carboxylic acid for tetrahydrofuran-3-carboxylic acid. LCMS (Method B): m/z 471.2 (M+H), retention time: 1.81 minutes; ¹H NMR (CDCl₃, 400 MHz) δ 8.17 (d, J=3.2 Hz, 1H), 7.86 (s, 1H), 7.78 (s, 1H), 7.72 (s, 1H), 6.04 (d, J=3.2 Hz, 1H), 5.00 (d, J=6.0 Hz, 1H), 4.70-4.65 (m, 1H), 4.49 (s, 2H), 4.99 (d, J=5.2 Hz, 2H), 3.96 (s, 3H), 2.98-2.85 (m, 2H), 2.32-2.23 (m, 2H), 1.81-1.67 (m, 5H).

### Example 79

### {4-[6,7-dichloro-4-(cyclopentylamino)phthalazin-1-yl]-3,6-dihydropyridin-1(2H)-yl}(1-methylpyrrolidin-3-yl)methanone

Example 79 was prepared using the procedure for the synthesis of Example 72, substituting 1-methylpyrrolidine-3-carboxylic acid for tetrahydrofuran-3-carboxylic acid. LCMS (Method B): m/z 474.2 (M+H), retention time: 1.81 minutes; ¹H NMR (CD₃OD, 400 MHz) δ 8.63 (s, 1H), 8.17 (d, J=3.2 Hz, 1H), 6.04 (d, J=8.0 Hz, 1H), 4.57-4.54 (m, 1H), 4.39-4.34 (m, 2H), 3.93-3.89 (m, 2H), 3.40-3.35 (m, 1H), 3.03-3.01 (m, 1H), 2.83-2.56 (m, 5H), 2.42 (s, 3H), 2.22-1.84 (m, 4H), 1.71-1.69 (m, 6H).

### Example 80

### 1-{4-[6,7-dichloro-4-(cyclopentylamino)phthalazin-1-yl]-3,6-dihydropyridin-1(2H)-yl}-2-methoxyethanone

Example 80 was prepared using the procedure for the synthesis of Example 72, substituting 2-methoxyacetic acid for tetrahydrofuran-3-carboxylic acid. LCMS (Method B): m/z 435.2 (M+H), retention time: 1.83 minutes; ¹H Wit (CD₃OD, 400 MHz) δ 8.51 (s, 1H), 8.07 (d, J=4.4 Hz, 1H), 5.91 (d, J=20.8 Hz, 1H), 4.47-4.43 (m, 1H), 4.23-4.17 (m, 4H), 3.81 (t, J=6.0 Hz, 1H), 3.69 (t, J=6.0 Hz, 1H), 3.35 (s, 3H), 2.98-2.94 (m, 1H), 2.58 (d, J=29.6 Hz, 2H), 2.12-2.05 (m, 2H), 1.73-1.58 (m, 6H).

### Example 81

### 3-({4-[6,7-dichloro-4-(cyclopentylamino)phthalazin-1-yl]-3,6-dihydropyridin-1(2H)-yl}carbonyl)cyclopentanone

Example 81 was prepared using the procedure for the synthesis of Example 72, substituting 3-oxocyclopentanecarboxylic acid for tetrahydrofuran-3-carboxylic acid. LCMS (Method B): m/z 473.2 (M+H), retention time: 2.05 minutes; ¹H NMR (DMSO-d₆, 400 MHz) δ 8.81 (s, 1H), 8.20 (d, J=5.2 Hz, 1H), 7.39 (d, J=6.8 Hz, 1H), 6.00 (s, 1H), 4.54-4.52 (m, 1H), 4.38-4.36 (m, 1H), 4.24-4.21 (m, 1H), 3.85-3.79 (m, 2H), 3.59-3.57 (m, 1H), 2.68-2.64 (m, 1H), 2.50-2.30 (m, 2H), 2.24-2.03 (m, 6H), 1.75-1.58 (m, 6H).

### Example 82

### 1-{4-[6,7-dichloro-4-(cyclopentylamino)phthalazin-1-yl]-3,6-dihydropyridin-1(2H)-yl}-2-(methylsulfonyl)ethanone

Example 82 was prepared using the procedure for the synthesis of Example 72, substituting 2-(methylsulfonyl)acetic acid for tetrahydrofuran-3-carboxylic acid. LCMS (Method B): m/z 483.1 (M+H), retention time: 2.00 minutes; ¹H NMR (DMSO-d₆, 400 MHz) δ 8.83 (s, 1H), 8.22 (d, J=17.2 Hz, 1H), 7.50 (brs, 1H), 5.97 (d, J=13.6 Hz, 1H), 4.59-4.51 (m, 3H), 4.35 (d, J=2.4 Hz, 1H), 4.26 (d, J=2.0 Hz, 1H), 3.84-3.79 (m, 2H), 3.15 (s, 3H), 2.68-2.50 (m, 2H), 2.07-2.03 (m, 2H), 1.75-1.57 (m, 7H).

### Example 83

### 3-{4-[6,7-dichloro-4-(cyclopentylamino)phthalazin-1-yl]-3,6-dihydropyridin-1(2H)-yl}-3-oxopropanamide

Example 83 was prepared using the procedure for the synthesis of Example 72, substituting 3-amino-3-oxopropanoic acid for tetrahydrofuran-3-carboxylic acid. LCMS (Method B): m/z 448.2 (M+H), retention time: 1.70 minutes; ¹H NMR (CD₃OD, 400 MHz) δ 8.51 (s, 1H), 8.09 (d, J=13.6 Hz, 1H), 5.91 (d, J=12.8 Hz, 1H), 4.46-4.43 (m, 1H) , 4.25 (d, J=2.4 Hz, 2H), 3.83 (t, J=5.6 Hz, 1H), 3.75 (t, J=5.6 Hz, 1H), 2.64-2.56 (m, 2H), 2.10-2.05 (m, 2H), 1.75-1.54 (m, 7H).

### Example 84

### {4-[6,7-dichloro-4-(cyclopentylamino)phthalazin-1-yl]-3,6-dihydropyridin-1(2H)-yl}(3-hydroxycyclopentyl)methanone

Example 84 was prepared using the procedure for the synthesis of Example 72, substituting 3-hydroxycyclopentanecarboxylic acid for tetrahydrofuran-3-carboxylic acid. LCMS (Method B): m/z 475.2 (M+H), retention time: 1.85 minutes; ¹H NMR (CD₃OD, 400 MHz) δ 8.51 (s, 1H), 8.06 (d, J=8.4 Hz, 1H), 5.94 (s, 1H), 4.49-4.47 (m, 1H), 4.30-4.16 (m, 3H), 3.83-3.80 (m, 2H), 2.62 (s, 1H), 2.53 (s, 1H), 2.10-2.05 (m, 3H), 1.90-1.59 (m, 11H).

### Determination of Biological Activity

Test compounds were dispensed by a Labcyte Echo in 384-well low volume assay plates with 3-fold serial dilutions from 50 μM to 0.00075 μM. 5 μL of SUV420H1 enzyme (Abbvie) at 90 nM was added to all wells except for the no enzyme control wells and incubated for 30 minutes at room temperature before 5 μL of Histone Peptide H4(8-30)me1 (Anaspec) at 5 μM and SAM at 4 μM was added to all wells. The plates were incubated in a humidified chamber overnight at room temp and then 10 μL of the detection reagent that contained RIPA buffer (50 mM Tris-HCl, pH 8.0, 150 mM sodium chloride, 1.0% Igepal CA-630 (NP-40), 0.5% sodium deoxycholate and 0.1% sodium dodecyl sulfate) (Sigma) with 2 nM of Terbium chelate anti S-adenosyl-L-homocysteine (SAH) (Abbvie) antibody and Oregon Green labeled SAH at 4 nM was added. The plates were further incubated for 4 hours at room temperature. The plates were read in a Perkin Elmer Envision using a TR-FRET protocol with Excitation set at 335 nm and Emission at 520 and 495 nm. To generate dose response curves the data is normalized to percent inhibition by setting the average of the plus and minus enzyme control wells to 0% and 100% inhibition respectively. The ICso values for the compounds are generated by fitting the normalized data with Accelrys Assay Explorer 3.3 to a sigmoidal curve model using linear regression, Y=(100*xⁿ)/(Kⁿ+xⁿ), where Y is the measured response, x is the compound concentration, n is the Hill Slope and K is the IC₅₀ and the lower and higher asymptotes are constrained to 0 and 100 respectively.

The IC₅₀ of the test compounds are presented in Table 1.

TABLE 1

| | TR-FRET SAH_SUV420H1 |
|---|---|
| Example Number | IC₅₀ (μM) |
| 1 | 0.361 |
| 2 | 28.9 |
| 4 | 0.0942 |
| 5 | 0.111 |
| 6 | 0.0386 |
| 7 | 0.0753 |
| 8 | 0.0871 |
| 9 | 0.373 |
| 10 | 48.4 |
| 11 | >50 |
| 12 | 0.827 |
| 13 | 2.63 |
| 14 | 19.1 |
| 15 | >50 |
| 16 | 8.35 |
| 17 | 7.38 |
| 18 | 0.525 |
| 19 | 0.163 |
| 20 | 0.415 |
| 21 | 1.46 |
| 22 | 1.01 |
| 23 | 1.51 |
| 24 | 1.28 |
| 25 | 1.1 |
| 26 | 0.389 |
| 27 | 1.32 |
| 28 | 0.572 |
| 29 | 0.47 |
| 30 | 1.05 |
| 31 | 2.74 |
| 32 | 11.2 |
| 33 | 1.24 |
| 34 | 2.73 |
| 35 | 0.808 |
| 36 | 0.173 |
| 37 | 0.0903 |
| 38 | 8.68 |
| 39 | 3.49 |
| 40 | 0.229 |
| 41 | 0.805 |
| 42 | 0.127 |
| 43 | 0.174 |
| 44 | 0.255 |
| 45 | 0.197 |
| 46 | 5.46 |
| 47 | 3.78 |
| 48 | 4.16 |
| 49 | 6.09 |
| 50 | 5.06 |
| 51 | 8.21 |
| 52 | 12.8 |
| 53 | 1.34 |
| 54 | 0.147 |
| 56 | 0.165 |
| 57 | 0.204 |
| 58 | 6.01 |
| 59 | 0.766 |
| 61 | 0.19 |
| 62 | 0.333 |
| 63 | 0.208 |
| 64 | 0.188 |
| 65 | 0.551 |
| 66 | 0.14 |
| 67 | 0.448 |
| 68 | 0.453 |
| 69 | 2.35 |
| 81 | 2.23 |
| 82 | 0.681 |
| 83 | 0.816 |
| 84 | 0.688 |

### H4K20me3 Cellular PD Assay

A high content microscopy-based PD assay was used to analyze the decrease of H4K20me3 upon inhibition of SUV420H1 cellular activity. PC-3 cells were plated at 3,000 cells per well in black Collagen I coated 96-well plates (Perkin Elmer, #6005810) in RPMI supplemented with 10% heat inactivated FBS, 1% sodium pyruvate and 1% non-essential amino acids in a 37° C. tissue culture incubator. At 24 hours post seeding, cells were treated with the indicated compounds for 72 hours in an 8-point dose response using half log dilutions from 10 to 0.0316 μM and then fixed in 2% formaldhyde (Polysciences, Inc., #04018) for 10 minutes at room temperature. Cells were washed in PBS and permeablized in 0.1% Triton X-100 for 10 minutes at room temperature. After blocking in 1% BSA (Sigma, #A7030) in PBS for 1 hour at room temperature, a mouse monoclonal antibody directed toward H4K20me3 (Santa Cruz, #sc-134216) in antibody dilution buffer (0.3% BSA in PBS) was added for 16 hours at 4° C. Cells were then washed 3 times in PBS and an Alexa Fluor 555 conjugated goat anti-mouse (Life Technologies #A-21424) secondary antibody was added for 1 hour at room temperature. Hoechst 33342 diluted 1:10000 (Life Technologies #H3570) was also added during the secondary antibody incubation. Cells were washed 3 times in PBS and stored in PBS at 4° C. Fluorescent images were acquired within 24 hours on a Cell Insight (Thermo Fisher Scientific) using the Target Activation algorithm. Objects were identified via nuclear Hoescht staining, and changes in average mean intensity in the fluorescent signals of H4K20me3 were quantified using the HCS View software platform (Thermo Fisher Scientific). EC₅₀ values were calculated using a sigmoidal fit of the concentration/inhibition response curves and presented in Table 2.

TABLE 2

| Example | H4K20me3 Inhibition |
|---|---|
| Number | EC₅₀ (μM) |
| 1 | 11.4 |
| 4 | 1.16 |
| 5 | 0.505 |
| 6 | 0.522 |
| 7 | 1.51 |
| 8 | 2.92 |
| 9 | 3.43 |
| 18 | 1.05 |
| 19 | 1.21 |
| 20 | 9.54 |
| 26 | 4.5 |
| 29 | 3.08 |
| 30 | 0.465 |
| 36 | 1.67 |
| 37 | 1.12 |
| 40 | 0.532 |
| 42 | 2.83 |
| 43 | 7.91 |
| 54 | >20 |
| 56 | 6.73 |
| 57 | 10.7 |
| 61 | 4.99 |
| 62 | 10.5 |
| 64 | 6.57 |
| 66 | 2.49 |
| 70 | 15.6 |
| 78 | 13.1 |

It is understood that the foregoing detailed description and accompanying examples are merely illustrative and are not to be taken as limitations upon the scope of the invention, which is defined solely by the appended claims and their equivalents. Various changes and modifications to the embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof.

We claim:

1. A compound of formula (I) or a pharmaceutically acceptable salt thereof, wherein 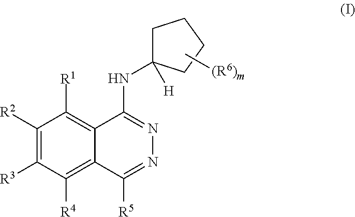
R¹ and R⁴ are each independently H, halogen, CN, C₁-C₃ alkyl, C₁-C₃ haloalkyl, —O—(C₁-C₃ alkyl), or —O—(C₁-C₃ haloalkyl);
R² and R³ are each independently halogen, CN, C₁-C₃ alkyl, C₁-C₃ haloalkyl, —O—(C₁-C₃ alkyl), or —O—(C₁-C₃ haloalkyl);
R⁵ is halogen, -G¹, —C(O)G², —C(O)O(R^{A}), or —C(O)N(R^{A})(R^{B});
G¹ is aryl, heteroaryl, or heterocycle, each of which is optionally substituted with 1, 2, or 3 R^{u} groups;
R^{u}, at each occurrence, is independently C₁-C₆ alkyl, C₂-C₆ alkenyl, C₂-C₆ alkynyl, halogen, C₁-C₆ haloalkyl, —CN, oxo, NO₂, —ORS, —OC(O)R^{k}, —OC(O)N(R^{j})₂, —S(O)₂R^{j}, —S(O)₂N(R^{j})₂, —C(O)R^{j}, —C(O)OR^{j}, —C(O)N(R^{j})₂, —N(R^{j})₂, —N(R^{j})C(O)R^{k}, —N(R^{j})S(O)₂R^{k}, —N(R^{j})C(O)O(R^{k}), —N(R^{j})C(O)N(R^{j})₂, -G^{1A}, (C₁-C₆ alkylenyl)-OR^{j}, —(C₁-C₆ alkylenyl)-OC(O)R^{k}, —(C₁-C6 alkylenyl)-OC(O)N(R^{j})₂, —(C₁-C6 alkylenyl)-SR^{j}, (C₁-C₆ alkylenyl)-S(O)₂R^{j}, —(C₁-C₆ alkylenyl)-S(O)₂N(R^{j})₂, —(C₁-C6 alkylenyl)-C(O)R^{j}, (C₁-C₆ alkylenyl)-C(O)OR^{j}, —(C₁-C₆ alkylenyl)-C(O)N(R^{j})₂, (C₁-C₆ alkylenyl)-N(R^{j})₂, (C₁-C₆ alkylenyl)-N(R^{j})C(O)R^{k}, —(C₁-C₆ alkylenyl)-N(R^{j})S(O)₂R^{k}, —(C₁-C₆ alkylenyl)-N(R^{j})C(O)O(R^{k}), —(C₁-C₆ alkylenyl)-N(R^{j})C(O)N(R^{j})₂, —(C₁-C₆ alkylenyl)-CN, or —(C₁-C₆ alkylenyl)-G^{1A};
R^{j}, at each occurrence, is independently hydrogen, C₁-C₆ alkyl, C₁-C₆ haloalkyl, -G^{1A}, —(C₁-C₆ alkylenyl)-OR^{m}, —(C₁-C₆ alkylenyl)-CN, (C₁-C₆ alkylenyl)-S(O)₂R^{m}, or —(C₁-C₆ alkylenyl)-C(O)N(R^{m})₂;
R^{k}, at each occurrence, is independently C₁-C₆ alkyl, C₁-C₆ haloalkyl, -G^{1A}, —(C₁-C₆ alkylenyl)-OR^{m}, —(C₁-C₆ alkylenyl)-CN, —(C₁-C₆ alkylenyl)-S(O)₂R^{m}, or —(C₁-C₆ alkylenyl)-C(O)N(R^{m})₂;
G^{1A}, at each occurrence, is independently aryl, cycloalkyl, heteroaryl, or heterocycle, each of which is optionally substituted with 1, 2, or 3 R^{v} groups;
G² is a monocyclic heterocycle which is optionally substituted with 1, 2, or 3 R^{v} groups;
R^{A}, at each occurrence, is independently H, C₁-C₆ alkyl, or C₁-C₆ haloalkyl;
R^{B} is H, C₁-C₆ alkyl, C₁-C₆ haloalkyl, C₁-C₆ hydroxyalkyl, phenyl, or monocyclic heteroaryl; wherein the phenyl and the monocyclic heteroaryl are optionally substituted with 1, 2, or 3 R^{v} groups;
R^{v}, at each occurrence, is independently C₁-C₆ alkyl, C₂-C₆ alkenyl, C₂-C₆ alkynyl, halogen, C₁-C₆ haloalkyl, —CN, oxo, NO₂, —OR^{m}, —OC(O)Rⁿ, —OC(O)N(R^{m})₂, —SR^{m}, —S(O)₂R^{m}, —S(O)₂N(R^{m})₂, —C(O)R^{m}, —C(O)OR^{m}, —C(O)N(R^{m})₂, —N(R^{m})₂, —N(R^{m})C(O)Rⁿ, —N(R^{m})S(O)₂Rⁿ, —N(R^{m})C(O)O(Rⁿ), —N(R^{m})C(O)N(Rⁿ)₂, —(C₁-C₆ alkylenyl)-OR^{m}, —(C₁-C₆ alkylenyl)-OC(O)Rⁿ, —(C₁-C₆ alkylenyl)-OC(O)N(R^{m})₂, —(C₁-C₆ alkylenyl)-SRⁿ, —(C₁-C₆ alkylenyl)-S(O)₂R^{m}, —(C₁-C₆ alkylenyl)-S(O)₂N(R^{m})₂, —(C₁-C₆ alkylenyl)-C(O)R^{m}, —(C₁-C₆ alkylenyl)-C(O)OR^{m}, —(C₁-C₆ alkylenyl)-C(O)N(R^{m})₂, —(C₁-C₆ alkylenyl)-N(R^{m})₂, —(C₁-C₆ alkylenyl)-N(R^{j})C(O)Rⁿ, —(C₁-C₆ alkylenyl)-N(R^{j})S(O)₂Rⁿ, —(C₁-C₆ alkylenyl)-N(R^{m})C(O)O(Rⁿ), —(C₁-C₆ alkylenyl)-N(R^{m})C(O)N(Rⁿ)₂, or —(C₁-C₆ alkylenyl)-CN;
R^{m}, at each occurrence, is independently hydrogen, C₁-C₆ alkyl, or C₁-C₆ haloalkyl;
Rⁿ, at each occurrence, is independently C₁-C₆ alkyl or C₁-C₆ haloalkyl;
R⁶ are optional substituents and at each occurrence, are each independently halogen, CN, C₁-C₃ alkyl, C₁-C₃ haloalkyl, —OH, —O—(C₁-C₃ alkyl), —O—(C₁-C₃ haloalkyl), or —S(O)₂—(C₁-C₃ alkyl); and
m is 0, 1, 2, or 3.

2. The compound of claim 1 or a pharmaceutically acceptable salt thereof, wherein R² and R³ are halogen.

3. The compound of claim 1 or a pharmaceutically acceptable salt thereof, wherein R² and R³ are Cl.

4. The compound of claim 1 or a pharmaceutically acceptable salt thereof, wherein R¹ and R⁴ are H.

5. The compound of claim 1 or a pharmaceutically acceptable salt thereof, wherein R⁵ is —C(O)G², —C(O)O(R^{A}), or —C(O)N(R^{A})(R^{B}).

6. The compound of claim 1 or a pharmaceutically acceptable salt thereof, wherein
R⁵ is -G¹ wherein G¹ is phenyl, C₅-C₆ heteroaryl, or C₄-C₆ heterocycle, each of which is optionally substituted with 1, 2, or 3 Ru^{u} groups.

7. The compound of claim 1 or a pharmaceutically acceptable salt thereof, wherein
R⁵ is —C(O)N(R^{A})(R^{B}).

8. The compound of claim 1 or a pharmaceutically acceptable salt thereof, wherein
R¹ and R⁴ are H, and
R² and R³ are halogen.

9. The compound of claim 1 or a pharmaceutically acceptable salt thereof, wherein
R² and R³ are halogen,
m is 0 or 1, and
R⁶ is halogen, CH₃, CH₂F, or —OH.

10. The compound of claim 1 or a pharmaceutically acceptable salt thereof, wherein
R² and R³ are halogen, and
R⁵ is -G¹, —C(O)G², —C(O)O(R^{A}), or —C(O)N(R^{A})(R^{B}).

11. The compound of claim 1 or a pharmaceutically acceptable salt thereof, wherein
R² and R³ are halogen, and
R⁵ is -G¹ wherein G¹ is phenyl, C₅-C₆ heteroaryl, or C₄-C₆ heterocycle, each of which is optionally substituted with 1, 2, or 3 R^{u} groups.

12. The compound of claim 1 or a pharmaceutically acceptable salt thereof, wherein
R² and R³ are halogen,
m is 0 or 1,
R⁶ is halogen, CH₃, CH₂F, or OH, and
R⁵ is -G¹ wherein G¹ is phenyl which is optionally substituted with 1, 2, or 3 R^{u} groups.

13. The compound of claim 1 or a pharmaceutically acceptable salt thereof, wherein
R² and R³ are halogen,
m is 0 or 1,
R⁶ is halogen, CH₃, CH₂F, or OH, and
R⁵ is -G¹ wherein G¹ is C₅-C₆ heteroaryl which is optionally substituted with 1, 2, or 3 R^{u} groups.

14. The compound of claim 1 or a pharmaceutically acceptable salt thereof, wherein
R² and R³ are halogen,
m is 0 or 1,
R⁶ is halogen, CH₃, CH₂F, or OH, and
R⁵ is -G¹ wherein G¹ is C₄-C₆ heterocycle which is optionally substituted with 1, 2, or 3 R^{u} groups.

15. The compound of claim 1 or a pharmaceutically acceptable salt thereof, wherein
R² and R³ are halogen,
m is 0 or 1,
R⁶ is halogen, CH₃, CH₂F, or OH, and
R⁵ is —C(O)G².

16. The compound of claim 1 or a pharmaceutically acceptable salt thereof, wherein
R² and R³ are halogen,
m is 0 or 1,
R⁶ is halogen, CH₃, CH₂F, or OH, and
R⁵ is —C(O)OR^{A}.

17. The compound of claim 1 or a pharmaceutically acceptable salt thereof, wherein
R² and R³ are halogen,
m is 0 or 1,
R⁶ is halogen, CH₃, CH₂F, or OH, and
R⁵ is —C(O)N(R^{A})(R^{B}).

18. The compound of claim 1 or a pharmaceutically acceptable salt thereof, wherein the compound is selected from the group consisting of
4,6,7-trichloro-N-cyclopentylphthalazin-1-amine;
6,7-dichloro-N-cyclopentyl-4-phenylphthalazin-1-amine;
6,7-dichloro-N-cyclopentyl-4-(pyridin-3-yl)phthalazin-1-amine;
6,7-dichloro-N-cyclopentyl-4-(1-methyl-1H-pyrazol-3-yl)phthalazin-1-amine;
6,7-dichloro-N-cyclopentyl-4-(1-methyl-1H-pyrazol-4-yl)phthalazin-1-amine;
6,7-dichloro-N-cyclopentyl-4-(pyridin-4-yl)phthalazin-1-amine;
6,7-dichloro-N-cyclopentyl-4-(thiophen-3-yl)phthalazin-1-amine;
6,7-dichloro-N-cyclopentyl-4-(piperidin-1-yl)phthalazin-1-amine;
6,7-dichloro-N-cyclopentyl-4-(4-methylpiperazin-1-yl)phthalazin-1-amine;
6,7-dichloro-N-cyclopentyl-4-(morpholin-4-yl)phthalazin-1-amine;
6,7-dichloro-N-cyclopentyl-4-(pyrrolidin-1-yl)phthalazin-1-amine;
6,7-dichloro-N-cyclopentyl-4-(2-fluoropyridin-4-yl)phthalazin-1-amine;
rac-(1R,3 S)-3-[(4,6,7-trichlorophthalazin-1-yl)amino]cyclopentanol;
6,7-dichloro-N-cyclopentyl-4-[4-(morpholin-4-ylmethyl)phenyl]phthalazin-1-amine;
6,7-dichloro-N-cyclopentyl-4-[2-(pyrrolidin-1-yl)pyridin-4-yl]phthalazin-1-amine;
6,7-dichloro-N-cyclopentyl-4-[2-(4-methylpiperazin-1-yl)pyridin-4-yl]phthalazin-1-amine;
6,7-dichloro-N-cyclopentyl-4-[2-(morpholin-4-yl)pyridin-4-yl]phthalazin-1-amine;
6,7-dichloro-N-cyclopentyl-4-[2-(piperazin-1-yl)pyridin-4-yl]phthalazin-1-amine;
6,7-dichloro-N-cyclopentyl-4-(6-fluoropyridin-3-yl)phthalazin-1-amine;
6,7-dichloro-N-cyclopentyl-4-[6-(dimethylamino)pyridin-3-yl]phthalazin-1-amine;
6,7-dichloro-N-cyclopentyl-4-[6-(4-methylpiperazin-1-yl)pyridin-3-yl]phthalazin-1-amine;
6,7-dichloro-N-cyclopentyl-4-[6-(morpholin-4-yl)pyridin-3-yl]phthalazin-1-amine;
tert-butyl 4-[6,7-dichloro-4-(cyclopentylamino)phthalazin-1-yl]-3,6-dihydropyridine-1(2H)-carboxylate;
6,7-dichloro-N-cyclopentyl-4-(1,2,3,6-tetrahydropyridin-4-yl)phthalazin-1-amine;
1-{4-[6,7-dichloro-4-(cyclopentylamino)phthalazin-1-yl]-3,6-dihydropyridin-1(2H)-yl}ethanone;
2-[{5-[6,7-dichloro-4-(cyclopentylamino)phthalazin-1-yl]pyridin-2-yl}(methyl)amino]ethanol;
6,7-dichloro-N-cyclopentyl-4-[6-(piperazin-1-yl)pyridin-3-yl]phthalazin-1-amine;
1-{5-[6,7-dichloro-4-(cyclopentylamino)phthalazin-1-yl]pyridin-2-yl}piperidin-4-ol;
1-{4-[6,7-dichloro-4-(cyclopentylamino)phthalazin-1-yl]pyridin-2-yl}piperidin-4-ol;
2-({5-[6,7-dichloro-4-(cyclopentylamino)phthalazin-1-yl]pyridin-2-yl}amino)ethanol;
6,7-dichloro-N-cyclopentyl-4-[6-(pyrrolidin-1-yl)pyridin-3-yl]phthalazin-1-amine;
6,7-dichloro-N-cyclopentyl-4-[2-(dimethylamino)pyridin-4-yl]phthalazin-1-amine;
4-[6-(3-aminopyrrolidin-1-yl)pyridin-3-yl]-6,7-dichloro-N-cyclopentylphthalazin-1-amine;
1-{5-[6,7-dichloro-4-(cyclopentylamino)phthalazin-1-yl]pyridin-2-yl}pyrrolidin-3-ol;
2-[{4-[6,7-dichloro-4-(cyclopentylamino)phthalazin-1-yl]pyridin-2-yl}(methyl)amino]ethanol;
1-{4-[6,7-dichloro-4-(cyclopentylamino)phthalazin-1-yl]pyridin-2-yl}pyrrolidin-3-ol;
methyl 6,7-dichloro-4-(cyclopentylamino)phthalazine-1-carboxylate;
6,7-dichloro-4-(cyclopentylamino)phthalazine-1-carboxylic acid;
2-({4-[6,7-dichloro-4-(cyclopentylamino)phthalazin-1-yl]pyridin-2-yl}amino)ethanol;
4-[2-(3-aminopyrrolidin-1-yl)pyridin-4-yl]-6,7-dichloro-N-cyclopentylphthalazin-1-amine;
6,7-dichloro-N-cyclopentyl-4-[6-(1,1-dioxidothiomorpholin-4-yl)pyridin-3-yl]phthalazin-1-amine;
6,7-dichloro-4-(cyclopentylamino)phthalazine-1-carboxamide;
tert-butyl {4-[6,7-dichloro-4-(cyclopentylamino)phthalazin-1-yl]-1H-pyrazol-1-yl}acetate;
{4-[6,7-dichloro-4-(cyclopentylamino)phthalazin-1-yl]-1H-pyrazol-1 -yl}acetic acid;
6,7-dichloro-4-(cyclopentylamino)-N-ethylphthalazine-1-carboxamide;
6,7-dichloro-4-(cyclopentylamino)-N-(2-hydroxyethyl)-N-methylphthalazine-1-carboxamide;
[6,7-dichloro-4-(cyclopentylamino)phthalazin-1-yl](pyrrolidin-1-yl)methanone;
[6,7-dichloro-4-(cyclopentylamino)phthalazin-1-yl](3-hydroxypyrrolidin-1-yl)methanone;
[6,7-dichloro-4-(cyclopentylamino)phthalazin-1-yl](4-methylpiperazin-1-yl)methanone;
[6,7-dichloro-4-(cyclopentylamino)phthalazin-1-yl](1,1-dioxidothiomorpholin-4-yl)methanone;
[6,7-dichloro-4-(cyclopentylamino)phthalazin-1-yl](morpholin-4-yl)methanone;
[6,7-dichloro-4-(cyclopentylamino)phthalazin-1-yl](4-hydroxypiperidin-1-yl)methanone;
6,7-dichloro-4-(cyclopentylamino)-N-phenylphthalazine-1-carboxamide;
6,7-dichloro-4-(cyclopentylamino)-N-(1-methyl-1H-pyrazol-4-yl)phthalazine-1-carboxamide;
2-{4-[6,7-dichloro-4-(cyclopentylamino)phthalazin-1-yl]-1H-pyrazol-1-yl}-N-ethylacetamide;
2-{4-[6,7-dichloro-4-(cyclopentylamino)phthalazin-1-yl]-1H-pyrazol-1-yl}-N-(2-hydroxyethyl)acetamide;
2-{4-[6,7-dichloro-4-(cyclopentylamino)phthalazin-1-yl]-1H-pyrazol-1-yl}-N-(2-hydroxyethyl)-N-methylacetamide;
2-{4-[6,7-dichloro-4-(cyclopentylamino)phthalazin-1-yl]-1H-pyrazol-1-yl}-1-(pyrrolidin-1-yl)ethanone;
2-{4-[6,7-dichloro-4-(cyclopentylamino)phthalazin-1-yl]-1H-pyrazol-1-yl}-1-(3-hydroxypyrrolidin-1-yl)ethanone;
2-{4-[6,7-dichloro-4-(cyclopentylamino)phthalazin-1-yl]-1H-pyrazol-1-yl}-1-(1,3-oxazolidin-3-yl)ethanone;
2-{4-[6,7-dichloro-4-(cyclopentylamino)phthalazin-1-yl]-1H-pyrazol-1-yl}-1-(4-methylpiperazin-1-yl)ethanone;
1-(4-acetylpiperazin-1-yl)-2-{4-[6,7-dichloro-4-(cyclopentylamino)phthalazin-1-yl]-1H-pyrazol-1-yl}ethanone;
2-{4-[6,7-dichloro-4-(cyclopentylamino)phthalazin-1-yl]-1H-pyrazol-1-yl}-1-(1,1-dioxidothiomorpholin-4-yl)ethanone;
2-{4-[6,7-dichloro-4-(cyclopentylamino)phthalazin-1-yl]-1H-pyrazol-1-yl}-1-(4-hydroxypiperidin-1-yl)ethanone;
2-{4-[6,7-dichloro-4-(cyclopentylamino)phthalazin-1-yl]-1H-pyrazol-1-yl}-1-(morpholin-4-yl)ethanone;
6,7-dichloro-4-(cyclopentylamino)-N-(2-hydroxyethyl)phthalazine-1-carboxamide;
6,7-dichloro-N-cyclopentyl-4-[1-(methylsulfonyl)-1,2,3,6-tetrahydropyridin-4-yl]phthalazin-1-amine;
6,7-dichloro-N-cyclopentyl-4-[1-(cyclopropylsulfonyl)-1,2,3,6-tetrahydropyridin-4-yl]phthalazin-1-amine;
6,7-dichloro-N-cyclopentyl-4-[1-(phenylsulfonyl)-1,2,3,6-tetrahydropyridin-4-yl]phthalazin-1-amine;
cyclopropyl{4-[6,7-dichloro-4-(cyclopentylamino)phthalazin-1-yl]-3,6-dihydropyridin-1(2H)-yl}methanone;
1-{4-[6,7-dichloro-4-(cyclopentylamino)phthalazin-1-yl]-3,6-dihydropyridin-1(2H)-yl}-2-hydroxypropan-1-one;
{4-[6,7-dichloro-4-(cyclopentylamino)phthalazin-1-yl]-3,6-dihydropyridin-1(2H)-yl}(tetrahydrofuran-3-yl)methanone;
3-{4-[6,7-dichloro-4-(cyclopentylamino)phthalazin-1-yl]-3,6-dihydropyridin-1(2H)-yl}-3-oxopropanenitrile;
{4-[6,7-dichloro-4-(cyclopentylamino)phthalazin-1-yl]-3,6-dihydropyridin-1(2H)-yl}(phenyl)methanone;
{4-[6,7-dichloro-4-(cyclopentylamino)phthalazin-1-yl]-3,6-dihydropyridin-1(2H)-yl}(2-methylphenyl)methanone;
{4-[6,7-dichloro-4-(cyclopentylamino)phthalazin-1-yl]-3,6-dihydropyridin-1(2H)-yl}(2,6-dimethylphenyl)methanone;
{4-[6,7-dichloro-4-(cyclopentylamino)phthalazin-1-yl]-3,6-dihydropyridin-1(2H)-yl}(pyridin-3-yl)methanone;
{4-[6,7-dichloro-4-(cyclopentylamino)phthalazin-1-yl]-3,6-dihydropyridin-1(2H)-yl}(1-methyl-1H-pyrazol-4-yl)methanone;
{4-[6,7-dichloro-4-(cyclopentylamino)phthalazin-1-yl]-3,6-dihydropyridin-1(2H)-yl}(1-methylpyrrolidin-3-yl)methanone;
1-{4-[6,7-dichloro-4-(cyclopentylamino)phthalazin-1-yl]-3,6-dihydropyridin-1(2H)-yl}-2-methoxyethanone;
3-({4-[6,7-dichloro-4-(cyclopentylamino)phthalazin-1-yl]-3,6-dihydropyridin-1(2H)-yl}carbonyl)cyclopentanone;
1-{4-[6,7-dichloro-4-(cyclopentylamino)phthalazin-1-yl]-3,6-dihydropyridin-1(2H)-yl}-2-(methylsulfonyl)ethanone;
3-{4-[6,7-dichloro-4-(cyclopentylamino)phthalazin-1-yl]-3,6-dihydropyridin-1(2H)-yl}-3-oxopropanamide; and
{4-[6,7-dichloro-4-(cyclopentylamino)phthalazin-1-yl]-3,6-dihydropyridin-1(2H)-yl}(3-hydroxycyclopentyl)methanone.

19. A pharmaceutical composition comprising a therapeutically effective amount of a compound of formula (I) according to claim 1, or a pharmaceutically acceptable salt thereof, in combination with a pharmaceutically acceptable carrier.

20. A kit for use in measuring the activity of SUV420H1or a fragment thereof in a biological sample in vitro or in vivo, comprising
(i) a first composition comprising a compound of formula (I) according to claim 1; and
(ii) instructions for:
(a) contacting the composition with the biological sample; and
(b) measuring activity of said SUV420H1 or a fragment thereof.

21. A method for modulating SUV420H1 in a membrane of a cell, comprising the step of contacting said cell with a compound of formula (I) according to claim for a pharmaceutically acceptable salt thereof.

22. A method of treating a condition, disease, or disorder in a subject implicated by SUV420H1 activity, comprising the step of administering to said subject a therapeutically effective amount of a compound of formula (I) according to claim 1 or a pharmaceutically acceptable salt thereof.
